(19) United States
(12) Patent Application Publication
(10) Pub. No.: US 2012/0202837 A1
(43) Pub. Date: Aug. 9, 2012

(54) SPIROCYCLIC PIPERIDINE DERIVATIVES USEFUL AS RENIN INHIBITORS

(76) Inventors: Austin Chen, Kirkland (CA); Reneé Aspiotis, Kirkland (CA); Daniel McKay, Kirkland (CA); Yongxin Han, Kirkland (CA); Pierre-André Fournier, Kirkland (CA)

(21) Appl. No.: 13/501,401

(22) PCT Filed: Oct. 12, 2010
(86) PCT No.: PCT/CA10/01632

§ 371 Date: Apr. 11, 2012

Related U.S. Application Data
(60) Provisional application No. 61/250,963, filed on Oct. 13, 2009.

(51) Int. Cl. A61K 31/438; C07D 491/20; A61P 9/12; A61P 9/00; A61P 27/02; A61P 27/06; A61P 25/22; C07D 491/107; A61P 9/04

(52) U.S. Cl. 514/278; 546/17; 546/18

(57) ABSTRACT

Renin inhibitors which are spirocyclic piperidine derivatives, of formula (I) and pharmaceutical compositions thereof useful in the treatment of cardiovascular diseases and renal insufficiency, or a stereoisomer thereof, or a pharmaceutically acceptable salt thereof, or a pharmaceutically acceptable salt of the stereoisomer thereof, wherein: n, for each instance in which it occurs, is independently 0, 1, or 2; W is a five- or six-membered saturated or unsaturated heterocyclic or carbocyclic monocyclic ring, A is (i) a five- or six-membered saturated or unsaturated heterocyclic or carbocyclic monocyclic ring or (ii) a first five- or six-membered saturated or unsaturated heterocyclic or carbocyclic ring which is fused to a second five- or six-membered saturated or unsaturated heterocyclic or carbocyclic ring, V is —(C═O)—, —CH₂— or ═CH—; U is a bond or —CH2-, or, when V iS ═CH—, U is —CH═; X is ═CH—, ═CF—, ═C(OR³)—, or —(C═O)—; and Y is ═CH—, ═CF—, ═N—, or, for the case when X is —(C═O)—, Y is —N(R³)—.

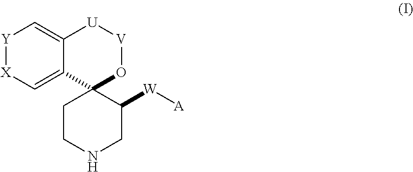

### JOINT RESEARCH AGREEMENT

The claimed invention was made pursuant to activities within the scope of a joint research agreement between Merck & Co., Inc. and Actelion Pharmaceuticals Ltd. executed on Dec. 4, 2003.

### FIELD OF THE INVENTION

The invention relates to novel renin inhibitors of general formula (I). The invention also concerns related aspects including pharmaceutical compositions containing one or more compounds of formula (I) and their use as renin inhibitors, particularly for the treatment of cardiovascular events and renal insufficiency.

### BACKGROUND OF THE INVENTION

In the renin-angiotensin system (RAS), biologically active angiotensin II (Ang II) is generated via a two-step mechanism. The highly specific renin enzyme initially cleaves angiotensinogen to angiotensin I (Ang I), which is then further processed to Ang II by the less specific angiotensin-converting enzyme (ACE). Ang II is currently known to act on four receptor subtypes, AT₁₋₄. AT₁ seems to transmit most of the known functions of Ang II, i.e., vasoconstriction, increased cardiac contractility, renal tubular sodium reabsorption, vascular and cardiac hypertrophy, etc. (see, e.g., Lévy, B. I., Circulation, 2004, 109, 8). AT₂₋₄ are less well-characterized; AT₂ may antagonize the effects of AT₁ (see, e.g., Porrello, E. R. et al., Frontiers in Bioscience, 2009, 14, 958).

Modulation of the RAS thus represents a major target for the treatment of cardiovascular diseases. ACE inhibitors and angiotensin receptor blockers (ARBs) have been used to treat hypertension. In addition, ACE inhibitors are in clinical use for renal protection (Kshirsagar, K. V. et al., American Journal of Kidney Diseases, 2000, 35, 695), the prevention of congestive heart failure (Konstam M. A. et al. Circulation, 1992, 6, 431) and treatment after myocardial infarction (Pfeffer, M. A. et al., N. Engl. J. Med., 1992, 327, 669).

Renin inhibitors present an attractive therapeutic approach due to the specificity of renin (Kleinert H. D., Cardiovasc. Drugs, 1995, 9, 645; McInnes, G. T., J. Human Hypertension, 2007, 21, 766). The only substrate known for renin is angiotensinogen, which can only be processed (under physiological conditions) by renin. By contrast, ACE cleaves bradykinin in addition to Ang I, and Ang I can also be cleaved by chymase, a serine protease (Husain A., J. Hypertens., 1993, 11, 1155). In some patients administration of ACE inhibitors leads to bradykinin accumulation, causing cough and potentially life-threatening angioneurotic edema (Israili Z. H. et al., Annals of Internal Medicine, 1992, 117, 234). Importantly, because chymase is not inhibited by ACE inhibitors, the formation of Ang II can still occur in patients treated with ACE inhibitors.

Blockade of the AT₁ receptor by ARBs such as losartan results in increased levels of circulating Ang II and it has been suggested that AT₂ receptor stimulation may be harmful in the longer term (see, e.g., Reudelhuber, T. L., Hypertension. 2005, 46, 1261).

As renin inhibitors would be expected to demonstrate a different pharmaceutical profile than ACE inhibitors and ARBs with regard to efficacy in blocking the RAS, they may represent an alternative to some of the more harmful aspects of these agents.

The compounds of the present invention inhibit renin and represent a novel structural class of renin inhibitors. These non-peptidic compounds are orally active and of low molecular weight. They are useful for any of those clinical indications in which renin inhibition may be desirable.

### SUMMARY OF THE INVENTION

The present invention is directed to piperidine-derivative compounds and pharmaceutically acceptable salts thereof.

The present invention further relates to processes for preparation of the compounds as well as pharmaceutical compositions containing one or more of said compounds in free form or in pharmaceutically acceptable salt form, together with one or more customary pharmaceutical excipient(s), as well as methods for inhibition of renin activity and of treatment for conditions in which renin inhibition may have a therapeutic effect. Such conditions include hypertension, congestive heart failure, cardiac hypertrophy, cardiac fibrosis, postinfarction cardiomyopathy, nephropathy, vasculopathy, neuropathy, restenosis following angioplasty, raised intra-ocular pressure, glaucoma, abnormal vascular growth, hyperaldosteronism, and anxiety states.

The present invention also relates to methods of inhibiting renin activity, wherein said method comprises the step of administering a compound according to formula (I) in an amount sufficient to provide an effective amount for renin inhibition in an organism.

### DETAILED DESCRIPTION OF THE INVENTION

The present invention is directed to compounds of Formula (I):

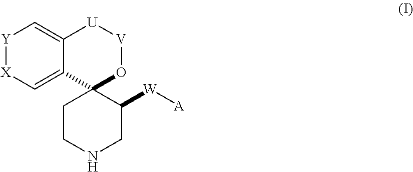

or a stereoisomer thereof, or a pharmaceutically acceptable salt thereof, or a pharmaceutically acceptable salt of the stereoisomer thereof, wherein:
n, for each instance in which it occurs, is independently 0, 1, or 2;
W is a five- or six-membered saturated or unsaturated heterocyclic or carbocyclic monocyclic ring,
wherein the heterocyclic ring contains 1-3 heteroatoms independently selected from N, O and S, wherein each N is optionally in the form of an oxide and each S is optionally in the form of an oxide selected from the group consisting of S(═O) and S(═O)₂,
wherein the heterocyclic or carbocyclic ring is optionally substituted by 1-4 radicals independently selected from the group consisting of:
   1) halogen,
   2) cyano,
   3) R¹,
   4) —(CH₂)₀₋₃C(O)R¹,
   5) —(CH₂)₀₋₃OR¹,
   6) —C(O)OR¹,
   7) —(CH₂)₁₋₃CN,
   8) —(CH₂)₁₋₃NHC(O)R¹,
   9) —(CH₂)₁₋₃NHC(O)OR¹,
   10) —(CH₂)₁₋₃NHC(O)(CH₂)₁₋₃OR¹,
      wherein substituents (3)-(10) can be further optionally substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or Z, wherein Z is morpholine, oxomorpholine, pyrrolidine, succinimide, acylmorpholine, or thiomorpholine 1,1-dioxide;

A is (i) a five- or six-membered saturated or unsaturated heterocyclic or carbocyclic monocyclic ring or (ii) a first five- or six-membered saturated or unsaturated heterocyclic or carbocyclic ring which is fused to a second five- or six-membered saturated or unsaturated heterocyclic or carbocyclic ring,
wherein the heterocyclic ring in (i), the first heterocyclic ring in (ii) which is fused to the second heterocyclic ring or carbocyclic ring, and the second heterocyclic ring in (ii) to which is fused the first heterocyclic or carbocyclic ring, contain 1-3 heteroatoms independently selected from N, O and S, wherein each N is optionally in the form of an oxide and each S is optionally in the form of an oxide selected from the group consisting of S(═O) and S(═O)₂,
wherein the heterocyclic ring and carbocyclic ring of (i), the first heterocyclic ring and first carbocyclic ring of (ii) and the second heterocyclic ring and second carbocyclic ring of (ii) are independently optionally substituted by 1-4 radicals independently selected from the group consisting of:
   1) halogen,
   2) cyano,
   3) R¹,
   4) —(CH₂)₀₋₃C(O)R¹,
   5) —(CH₂)₀₋₃OR¹,
   6) —C(O)OR¹,
   7) —(CH₂)₁₋₃CN,
   8) —(CH₂)₁₋₃NHC(O)R¹,
   9) —(CH₂)₁₋₃NHC(O)OR¹,
   10) —(CH₂)₁₋₃NHC(O)(CH₂)₁₋₃OR¹,
      wherein substituents (3)-(10) can be further optionally substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or Z, wherein Z is morpholine, oxomorpholine, pyrrolidine, succinimide, acylmorpholine, or thiomorpholine 1,1-dioxide;
R² is hydrogen, C₁₋₄ alkyl, C₁₋₄ alkanoyl or C₃₋₆ cycloalkyl, wherein said C₁₋₄ alkyl, C₁₋₄ alkanoyl or C₃₋₆ cycloalkyl group can be independently substituted with 1-3 halogens;

R¹ and R³ are independently selected from the group consisting of R³ hydrogen, C₁₋₄ alkyl, C₂₋₆ alkenyl and C₃₋₆ cycloalkyl, wherein said C₁₋₄ alkyl, C₂₋₆ alkenyl and C₃₋₆ cycloalkyl group can be independently substituted with 1-3 halogens;
V is —(C═O)—, —CH₂— or ═CH—;
U is
   a bond or —CH2-, or,
   when V is ═CH—, U is —CH═;
X is ═CH—, ═CF—, ═C(OR³)—, or —(C═O)—; and
Y is ═CH—, ═CF—, ═N—, or, for the case when X is —(C═O)—, Y is —N(R³)—.

In a preferred embodiment, a compound of formula (Ia) is provided:

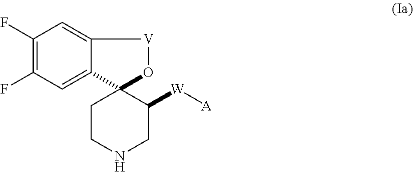

and all other variables are as previously defined.

In another preferred embodiment, W is selected from the group consisting of

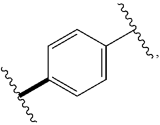

which is unsubstituted or substituted with C₁₋₄ alkyl, and

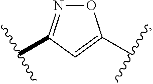

which is unsubstituted or substituted with halogen, and all other variables are as previously defined.

In another preferred embodiment, A is substituted or unsubstituted aryl, and all other variables are as previously defined.

In another preferred embodiment, A is aryl, which is unsubstituted or substituted with 1-4 radicals selected from the group consisting of:
1) —CH₂OC(CH₃)₃,
2) —CH₂OH,
3) —CH₂CN,
4) —CH₂CH₂NHC(O)CH₃,
5) —(CH₂)₃OCH₃,
6) —CH₂CH₂C(O)CH₃,
7) —CH₃,
8) —CH₂CH₂NHC(O)CH₂CH₃,
9) —CH₂CH₂NHC(O)OCH₃, and
10) —CH₂CH₂NHC(O)CH₂OH,
   and all other variables are as previously defined.

Specific examples of compounds of formula I, and pharmaceutically acceptable salts thereof, include the following:
rac-(1R,3′S)3′-[2′-(tert-Butoxymethyl)-3-methyl-4-biphenylyl]-5,6-Difluoro-3H-spiro[2-benzofuran-1,4′-piperidine] (Example 1)
rac-{4′-[(1R,3′S)-5,6-Difluoro-3H-spiro[2-benzofuran-1,4′-piperidin]-3′-yl]-3′-methyl-2-biphenylyl}methanol (Example 2)
rac-{4′-[(1R,3′S)-5,6-Difluoro-3H-spiro[2-benzofuran-1,4′-piperidin]-3′-yl]-3′-methyl-2-biphenylyl}acetonitrile (Example 3)
rac-N-(2-{4′-[(1R,3′S)-5,6-Difluoro-3H-spiro[2-benzofuran-1,4′-piperidin]-3′-yl]-3′-methyl-2-biphenylyl}ethyl)acetamide (Example 4)
rac-(1R,3′S)-3′-[2′-(3-Methoxypropyl)-3-methyl-4-biphenylyl]-5-methyl-3,5-dihydro-6H-spiro[furo[3,4-c]pyridine-1,4′-piperidin]-6-one (Example 5)
rac-(1R,3′S)-5-methyl-3′-[3-methyl-2′-(3-oxobutyl)-4-biphenylyl]-3,5-dihydro-6H-spiro[furo[3,4-c]pyridine-1,4′-piperidin]-6-one (Example 6)
rac-N-(2-{4′-[(1R,3′S)-5,6-Difluoro-3H-spiro[2-benzofuran-1,4′-piperidin]-3′-yl]-3′,6-dimethyl-2-biphenylyl}ethyl)acetamide (Example 7)
N-(2-{4′-[(1R,3′S)-5,6-Difluoro-3-oxo-3H-spiro[2-benzofuran-1,4′-piperidin]-3′-yl]-3′-methyl-2-biphenylyl}ethyl)acetamide (Example 8)
N-(2-{4′-[(1R,3′S)-5,6-Difluoro-3-oxo-3H-spiro[2-benzofuran-1,4′-piperidin]-3′-yl]-3′-methyl-2-biphenylyl}ethyl)propanamide (Example 9)
Methyl (2-{4′-[(1R,3′S)-5,6-Difluoro-3-oxo-3H-spiro[2-benzofuran-1,4′-piperidin]-3′-yl]-3′-methyl-2-biphenylyl}ethyl)carbamate (Example 10)
N-(2-{4′-[(1R,3′S)-5,6-Difluoro-3-oxo-3H-spiro[2-benzofuran-1,4′-piperidin]-3′-yl]-3′-methyl-2-biphenylyl}ethyl)-2-hydroxyacetamide (Example 11)
N-(2-{4′-[(1R,3′S)-5-fluoro-6-methoxy-3-oxo-3H-spiro[2-benzofuran-1,4′-piperidin]-3′-yl]-3′-methyl-2-biphenylyl}ethyl)-2-hydroxyacetamide (Example 12)
N-[2-(2-(4-Bromo-3-[(1R,3′R)-5,6-difluoro-3H-spiro[2-benzofuran-1,4′-piperidin]-3′-yl]-5-isoxazolyl)phenyl)ethyl]acetamide (Example 13)

The general terms used hereinbefore in Formula (I) and hereinafter have, within this disclosure, the following meanings, unless otherwise indicated. Where the plural form is used for compounds, salts, pharmaceutical compositions, diseases and the like, this is intended to mean also a single compound, salt, or the like.

Structural depictions of compounds may show a terminal methyl group as “—CH₃”, “CH₃”, “-Me”, “Me” or “ [symbol] ” (i.e., these have equivalent meanings). A terminal ethyl group may be depicted as “—CH₂CH₃”, “CH₂CH₃”, “-Et”, “Et” or “ [symbol] ̂” (i.e., these have equivalent meanings).

The term “alkyl”, alone or in combination with other groups, unless indicated otherwise, means saturated, straight and branched chain groups with one to six carbon atoms (which may be represented by “C₁₋₆ alkyl”). When the intended meaning is other than this, for example, when the number of carbon atoms is in the range of one to four carbon atoms, this meaning is represented in like fashion as “C₁₋₄ alkyl”. Example of alkyl groups are methyl, ethyl, n-propyl, iso-propyl, n-butyl, n-pentyl, n-hexyl and etc.

The term “alkenyl”, alone or in combination with other groups, unless indicated otherwise, means unsaturated (i.e. having at least one double bond) straight and branch chain groups with two to six carbon atoms (which may be represented by C₂₋₆ alkenyl). When the intended meaning is other than this, for example, when the number of carbon atoms is in the range of two to four carbon atoms, this meaning is represented in like fashion as C₂₋₄ alkenyl.

The term “alkoxy”, alone or in combination with other groups, refers to an R-0 group, wherein R is an alkyl group. Example of alkoxy groups are methoxy, ethoxy, propoxy, iso-propoxy, isobutoxy, tert-butoxy and etc.

The term “halogen” means fluorine, chlorine, bromine or iodine. In specific embodiments, halogen is fluorine, chlorine or bromine. In particular embodiments, halogen is fluorine or chlorine.

The term “cycloalkyl”, alone or in combination with other groups, unless indicated otherwise, means a saturated cyclic hydrocarbon ring system with three to six carbon atoms, e.g. cyclopropyl, cyclobutyl, cyclopentyl or cyclohexyl. This may be represented by “C₃₋₆ cycloalkyl”.

The term “alkanoyl”, alone or in combination with other groups, unless indicated otherwise, refers to saturated, straight and branched chain groups with one to six carbon atoms with one of the said, unbranching carbon being a carbonyl group (i.e. C═O). This may be represented by “C₁₋₆ alkanoyl”. Examples of alkanoyl groups are —(C═O)H, —(C═O)CH₃, —CH₂(C═O)H, —(C═O)CH₂CH₃, —CH₂(C═O)CH₃ and etc.

The term “carboxy”, alone or in combination with other groups, unless indicated otherwise, refers to —(C═O)O—.

The term “carbocycle” (and variations thereof such as “carbocyclic” or “carbocyclyl”) are used as herein, unless otherwise indicated, refers to a C₃ to C₈ monocyclic saturated or unsaturated ring. The carbocycle may be attached to the rest of the molecule at any carbon atom which results in a stable compound. Saturated carbocyclic rings are also referred to as cycloalkyl rings.

The term “monocycle” (and variations thereof such as “monocyclic”) as used herein refers to a single ring which may be substituted or unsubstituted with one or more substituents as described herein.

The term heterocycle” (and variations thereof such as “heterocyclic” or “heterocyclyl”) broadly refers to a stable four- to eight-membered, saturated or unsaturated monocyclic ring which contains one or more heteroatoms selected from N, O, and S and a balance of carbon atoms); wherein any one or more of the nitrogen and sulfur atoms is optionally oxidized, and any one or more of the nitrogen heteroatoms is optionally quaternized. Unless otherwise specified, when the heterocyclic ring has substituents, it is understood that the substituents may be attached to any atom in the ring, whether a heteroatom or a carbon atom, provided that a stable chemical structure results.

The term “aryl”, alone or in combination, relates to a phenyl, naphthyl or indanyl group. In specific embodiments, the “aryl” is phenyl.

The term “heteroaryl”, alone or in combination, means six-membered aromatic rings containing one to four nitrogen atoms; benzofused six-membered aromatic rings containing one to four nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing two heteroatoms independently selected from oxygen, nitrogen and sulfur and benzofused derivatives of such rings; five-membered aromatic rings containing three nitrogen atoms and benzofused derivatives there of; a tetrazolyl ring, a thiazinyl ring; or coumarinyl. Examples of such ring systems are furanyl, thienyl, pyrrolyl, pyridinyl, pyrimidinyl, indolyl, quinolinyl, isoquinolinyl, imidazolyl, triazinyl, thiazolyl, isothiazolyl, pyridazinyl, pyrazolyl, oxazolyl, isoxazolyl, benzothienyl, quinazolinyl, quinoxalinyl and etc.

The present invention also encompasses a pharmaceutical formulation comprising a pharmaceutically acceptable carrier and the compound of Formula (I) or a pharmaceutically acceptable crystal form or hydrate thereof.

In the compounds of generic Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula (I). For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula (I) can be prepared without undue experimentation by conventional techniques well known to those skilled in the art or by processes analogous to those described in the Schemes and Examples herein using appropriate isotopically-enriched reagents and/or intermediates.

Renin inhibitors, such as those disclosed herein, can be used for the treatment of essential hypertension. Compounds disclosed here are orally-bioavailable and thus should ideally be dosed orally. Alternative modes of administration such as through skin (e.g. transdermal), mucosal membranes (e.g. inhaler, lozenge, suppository) can also be employed when appropriate.

Because the compounds of the present invention inhibit renin, they are useful for blood pressure regulation and indications in which renin inhibition may be useful. Such indications include reduction of intra-ocular pressure, treatment of hypertension, congestive heart failure, cardiac hypertrophy, cardiac fibrosis, cardiomyopathy post-infarction, complications resulting from diabetes, such as nephropathy, vasculopathy and neuropathy, diseases of the coronary vessels, restenosis following angioplasty, raised intra-ocular pressure, glaucoma, abnormal vascular growth, hyperaldosteronism, anxiety states and cognitive disorders.

Compounds of Formula (I) or the above-mentioned pharmaceutical compositions are also of use in combination with other pharmacologically active compounds comprising ACE-inhibitors, neutral endopeptidase inhibitors, angiotensin II receptor antagonists, endothelin receptors antagonists, vasodilators, calcium antagonists, potassium activators, diuretics, sympatholytics, beta-adrenergic antagonists, alpha-adrenergic antagonists or with other drugs beneficial for the prevention or the treatment of the above-mentioned diseases.

Compounds of Formula (I), optionally in the form of a salt, can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, mucosally (including sublingual, buccal, rectal, nasal or vaginal administrations), parenterally (including subcutaneous injection, bolus injection, intra-arterial, intravenous, intramuscular, intrasternal injection or infusion administration techniques), by inhalation spray, transdermal, such as passive or iontophoretic delivery, or topical administration, in the form of a unit dosage of a pharmaceutical composition containing an effective amount of the compound and conventional non-toxic pharmaceutically-acceptable carriers, adjuvants and vehicles. Examples of dosage forms include, but are not limited to: tablets, caplets, capsules, such as soft elastic gelatin capsules, cachets, troches, lozenges, dispersions, suppositories, ointments, cataplasms (poultices), pastes, powders, dressings, creams, plasters, solutions, patches, aerosols (e.g., nasal sprays or inhalers), gels, liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs, liquid dosage forms suitable for parenteral administration to a patient, and sterile solids (e.g., crystalline or amorphous solids) that can be reconstituted to provide liquid dosage forms suitable for parenteral administration to a patient. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990.

### Methods of Synthesis

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2.sup.nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes and examples are merely illustrative of some methods by which the compounds of the present invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specifically stated otherwise, the experimental procedures were performed under the following conditions. Evaporation of solvent was carried out using a rotary evaporator under reduced pressure (600-4000 pascals: 4.5-30 mm Hg) with a bath temperature of up to 60° C. Reactions are typically run under nitrogen atmosphere at ambient temperature if not otherwise mentioned. Anhydrous solvent such as THF, DMF, Et₂O, DME and toluene are commercial grade. Reagents are commercial grade and were used without further purification. Flash chromatography is run on silica gel (230-400 mesh). The course of the reaction was followed by either thin layer chromatography (TLC) or nuclear magnetic resonance (NMR) spectrometry and reaction times given are for illustration only. The structure and purity of all final products were ascertained by TLC, mass spectrometry, ¹H NMR and high-pressure liquid chromatography (HPLC). Chemical symbols have their usual meanings. The following abbreviations have also been used: v (volume), w (weight), b.p. (boiling point), m.p. (melting point), L (liter(s)), mL (milliliter(s)), g (gram(s)), mg (milligram(s)), mol (mole(s)), mmol (millimole(s)), eq. (equivalent(s)). Unless otherwise specified, all variables mentioned below have the meanings as provided above. In the schemes, ring structures having fixed radicals or variable radicals attached to ring atoms are represented by a ring variable letter (either “A” or “W”), with a ring surrounding the ring variable letter, and bonds extending from the ring to the radical or variable radical. For example, the following depiction

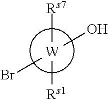

represents ring variable W to which is attached fixed radicals —Br and —OH, and variable radicals R^{s7} and R^{s1}.

Generally, compounds of the present invention can be prepared via the initial palladium-catalyzed C-arylation of the sodium enolate generated in situ from commercially available tert-butyl 4-oxo-1-piperidinecarboxylate (I). The biaryl bromide II used in this transformation can itself be readily assembled from, for example, palladium-catalyzed cross coupling of a simple aryl iodide III with metalated arene IV. The M in IV can, for example, be tin (Stille), boron (Suzuki), magnesium (Kumada) or silicon (Hiyama). From aryl ketone V, treatment with an appropriately functionalized Grignard reagent VI would then furnish tertiary alcohol VII. After the unmasking of a handle suitable for spirocyclization (PG=protecting group) to afford VIII (FG=functional group), subsequent intramolecular ring closure would then furnish spirocycle IX. Further functional group manipulations of the various pendant R groups using simple chemical transformations (e.g. acylation, alkylation and etc.) followed by BOC removal under typical conditions would reveal piperidine X (Scheme 1).

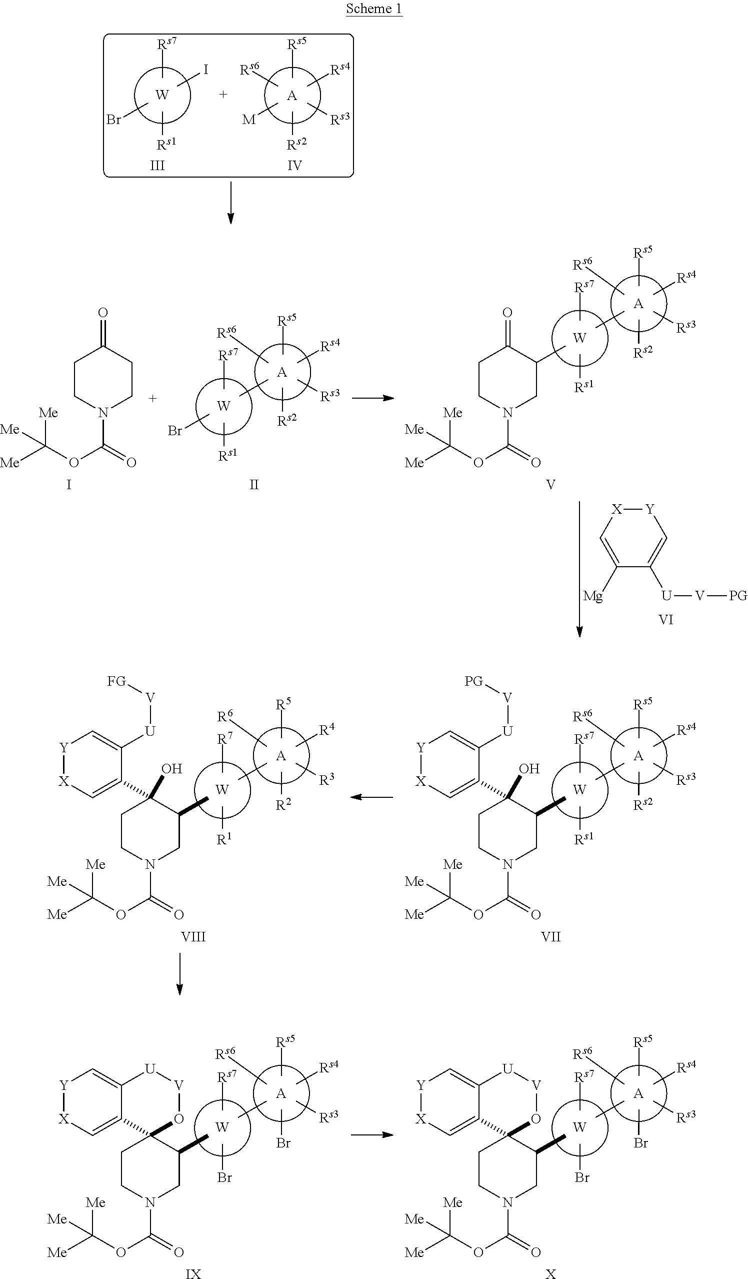

Alternatively, some compounds of the present invention can more preferably be accessed via the metal-promoted coupling of triflate XI with metalated arene IV. Triflate XI could in turn be assembled from commercially available tert-butyl 4-oxo-1-piperidinecarboxylate (I). In the forward direction, C-arylation of I with aryl bromide XII under typical conditions would afford, after protection of the free phenol, ketone XIII Spirolactonization with commercially available 2-bromo-4,5-difluorobenzoic acid (XIV) would then furnish spirolactone XV. Removal of the phenol protecting group (PG) in spirolactone XV and subsequent triflation would produce the requisite triflate XI. As before, further functional group manipulations of the various pendant R groups in spirolactone XVI using simple chemical transformations (e.g. acylation, alkylation and etc.) followed by BOC removal would reveal piperidine XVII (Scheme 2).

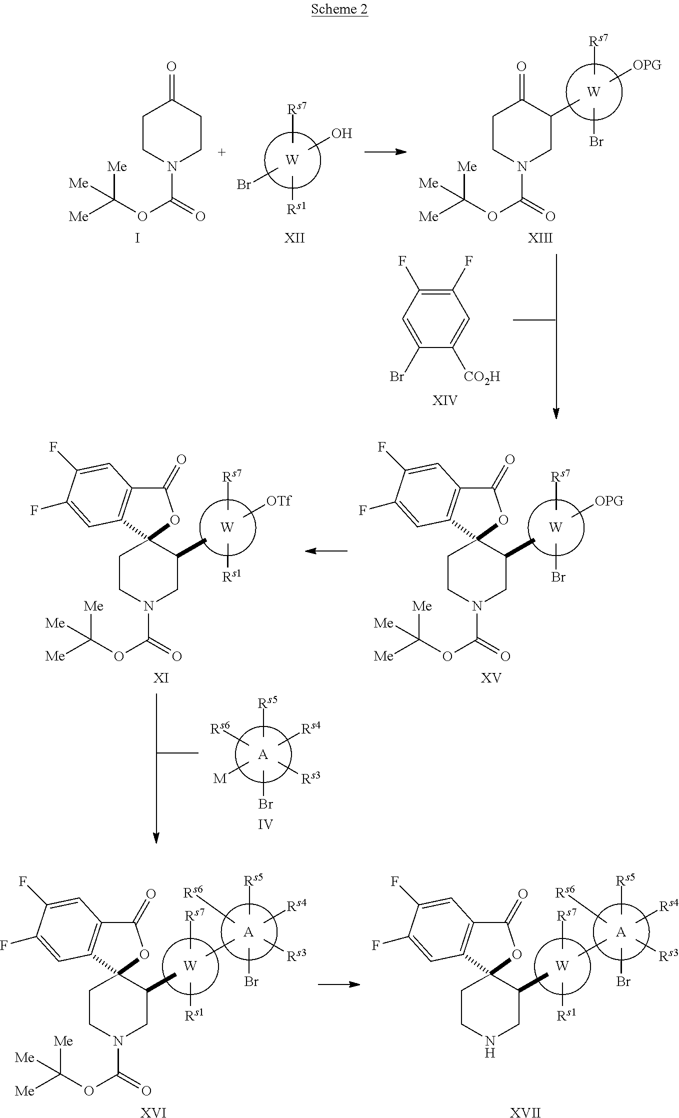

In instances where ring “W” is a 5-membered heteroaromatic ring such as, for example, an isoxazole, an alternate synthetic approach could be envisioned. Starting from known boronate XVIII, its palladium-mediated coupling with appropriately functionalized aryl bromide XIX would produce ester XX. Deprotection would trigger a spirocyclization event to afford spirocycle XXI. A subsequent oxidation state re-adjustment followed by the requisite [3+2] cyclization with an appropriately functionalized arene XXII (FG=functional group) would furnish heterobiaryl IX where ring “W” is the desired 5-membered heteroaromatic ring. As an illustrative example, if “W” is an isoxazole, the oxidative state re-adjustment of XXI would involve reduction of the ester to an aldehyde and its conversion to an oxime and the FG of arene XXII would be an acetylene. Finally, similar end game sequence as described previously would complete the synthesis (Scheme 3).

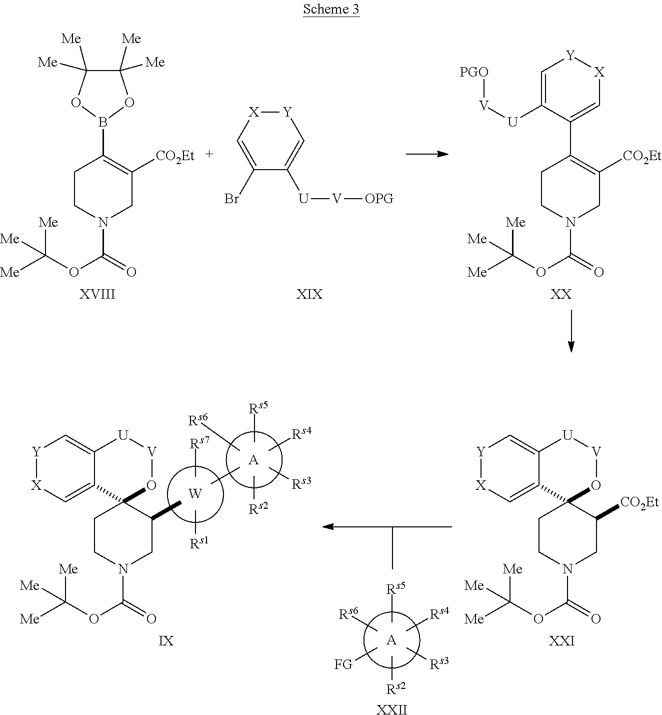

In Schemes 1, 2 and 3, variables R^{s1} and R^{s7} are independently selected from the group consisting of
1) halogen,
2) cyano,
3) R¹,
4) —(CH₂)₀₋₃C(O)R¹,
5) —(CH₂)₀₋₃OR¹,
6) —C(O)OR¹,
7) —(CH₂)₁₋₃CN,
8) —(CH₂)₁₋₃NHC(O)R¹,
9) —(CH₂)₁₋₃NHC(O)OR¹,
10) —(CH₂)₁₋₃NHC(O)(CH₂)₁₋₃OR¹, and
11) hydrogen,
   wherein substituents (3)-(10) can be further optionally substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or Z, wherein Z is morpholine, oxomorpholine, pyrrolidine, succinimide, acylmorpholine, or thiomorpholine 1,1-dioxide.

In Schemes 1, 2 and 3, variables R^{s2}, R^{s3}, R^{s4}, R^{s5}, and R^{s6} are independently selected from the group consisting of
1) halogen,
2) cyano,
3) R¹,
4) —(CH₂)₀₋₃C(O)R¹,
5) —(CH₂)₀₋₃OR¹,
6) —C(O)OR¹,
7) —(CH₂)₁₋₃CN,
8) —(CH₂)₁₋₃NHC(O)R¹,
9) —(CH₂)₁₋₃NHC(O)OR¹,
10) —(CH₂)₁₋₃NHC(O)(CH₂)₁₋₃ OR¹, and
11) hydrogen,
   wherein substituents (3)-(10) can be further optionally substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or Z,
      wherein Z is morpholine, oxomorpholine, pyrrolidine, succinimide, acylmorpholine, or thiomorpholine 1,1-dioxide.

Representative compounds of the invention can be synthesized in accordance with the general synthetic scheme above and are illustrated in the examples that follow. The methods for preparing the various starting materials used in the schemes and examples are well within the knowledge of persons skilled in the art.

The Grignard reagents in Table 1 were synthesized as follows.

TABLE 1

| COMPOUND | STRUCTURE |
|---|---|
| GRIGNARD 1 | 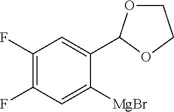 |
| GRIGNARD 1 | 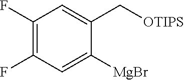 |
| GRIGNARD 2 | 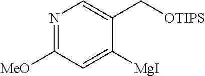 |

### Grignard 1

### Bromo[2-(1,3-dioxan-2-yl)-4,5-difluorophenyl]magnesium

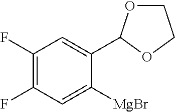

### Step 1: (2-Bromo-4,5-difluorophenyl)methanol

To a refluxing THF solution (0.42 M) of 2-bromo-4,5-difluorobenzoic acid (1 eq.) was added dropwise neat borane-methyl sulfide complex (1.25 eq.) over a period of 30 min. The resulting solution was heated at reflux for another 2 h before the crude reaction mixture was diluted with ether and carefully quenched with 10% aq. HCl. The aqueous wash was then separated and back-extracted with ether. The combined organic extracts were then washed sequentially with 1 N aq. NaOH, water and brine. The organic extract was dried over Na₂SO₄, filtered and the filtrate concentrated in vacuo to furnish the title compound as a white solid.

### Step 2: 2-Bromo-4,5-difluorobenzaldehyde

To a dichloromethane suspension (0.2 M) of (2-bromo-4,5-difluorophenyl)methanol (1 eq.) from the previous step and sodium bicarbonate (1.5 eq.) was added DMP (1.2 eq.) in one rapid portion. The resulting suspension was stirred at RT for 2 h before it was diluted with ether and washed sequentially with 5% aq. NaHSO₃, 1 N aq. NaOH, water and brine. The organic extract was then dried over Na₂SO₄, filtered through a pad of SiO₂ and the filtrate concentrated in vacuo. Purification of the crude product thus obtained by way of column chromatography (SiO₂, Hex→1:1 (v/v) Hex: EtOAc) afforded the title compound as a white solid.

### Step 3: 2-(2-Bromo-4,5-difluorophenyl)-1,3-dioxolane

To a reaction vessel containing a benzene solution (0.12 M) of 2-bromo-4,5-difluorobenzaldehyde (1 eq.) from the previous step, 1,2-ethanediol (1 eq.) and TsOH (0.05 eq.) was attached a Dean-Stark apparatus. The reaction mixture was then heated at reflux. After 20 h the reaction mixture was cooled to RT, diluted with hexanes and ether, and washed sequentially with 1 N aq. NaOH, water and brine. The organic extract was then dried over Na₂SO₄, filtered through a pad of SiO₂ and the filtrate concentrated in vacuo. Purification of the crude product thus obtained by way of column chromatography (SiO₂, Hex→1:1 (v/v) Hex: EtOAc) afforded the title compound as a pale yellow oil that solidified upon standing.

### Step 4: Grignard 1

2-(2-Bromo-4,5-difluorophenyl)-1,3-dioxolane (0.2 M THF solution, 1 eq.) from the previous step was added to a 1 M THF suspension of Rieke magnesium (2 eq.) at a rate as to keep the internal reaction temperature below 45° C. The resulting dark suspension was then allowed to stir at RT for 3 h before unreacted Rieke magnesium was removed via filtration. The concentration of the title compound in THF thus obtained was determined via titration to be 0.055 M.

### Grignard 2

Iodo(2-methoxy-5-{[(triisopropylsilyl)oxy]methyl}-4-pyridinyl)magnesium

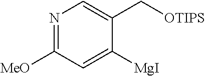

### Step 1: (4-Iodo-6-methoxy-3-pyridinyl)methanol

To a THF solution (0.26 M) of trimethylethylenediamine (1.2 eq.) was added nBuLi (2.5 M cyclohexane solution, 1.1 eq.) dropwise at −78° C. After 15 min, 6-methoxynicotinaldehyde (1 eq.) was then added in one rapid portion. The resulting solution was stirred at −78° C. for another 15 min before nBuLi (2.5 M cyclohexane solution, 2 eq.) was added dropwise over 10 min. The reaction mixture was then allowed to warm slowly to −40° C. and allowed to stir at −40° C. for another 2 h. The reaction mixture was re-cooled to −78° C. before freshly re-crystallized 1,2-diiodoethane (2.1 eq.) was added. Finally, the cooling bath was removed and the resulting red suspension was allowed to stir at RT for 16 h. The reaction mixture was then diluted with ether and quenched with sat. aq. NaHSO₃. The organic layer was separated and washed further with 1 N aq. NaOH, water and brine. The organic extract was then dried over Na₂SO₄, filtered and the filtrate concentrated in vacuo. Purification of the crude product thus obtained by way of column chromatography (SiO₂, 9:1 (v/v) Hex:EtOAc→3:7 (v/v) Hex: EtOAc) afforded the title compound as a white solid.

### Step 2: 4-Iodo-2-methoxy-5-{[(triisopropylsilyl)oxy]methyl}pyridine

To a DMF solution (0.2 M) of (4-iodo-6-methoxy-3-pyridinyl)methanol (1 eq.) from the previous step and imidazole (1.5 eq.) was added chlorotriisopropylsilane (1.2 eq.). The resulting solution was allowed to stir at RT for 24 h before it was diluted with ether and washed with water and brine. The organic extract was dried over Na₂SO₄, filtered and the filtrate concentrated in vacuo. Purification of the crude product thus obtained by way of column chromatography (SiO₂, 9:1 (v/v) Hex: EtOAc→3:7 (v/v) Hex: EtOAc) afforded the title compound as a colorless oil.

### Step 3: Grignard 2

To a THF solution (0.38 M) of 4-iodo-2-methoxy-5-{[(triisopropylsilyl)oxy]methyl}pyridine (1 eq.) from the previous step was added isopropylmagnesium chloride (2 M in THF, 1 eq.). The resulting solution was stirred at RT for 30 min. The concentration of the title compound in THF thus obtained was determined via titration to be 0.032 M.

### Grignard 3

### Bromo(4,5-difluoro-2-{[(triisopropylsilyl)oxy]methyl}phenyl)magnesium

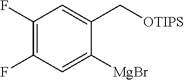

### Step 1: [(2-Bromo-4,5-difluorobenzyl)oxy](triisopropyl)silane

To a DMF solution (0.15 M) of (2-bromo-4,5-difluorophenyl)methanol (1 eq., Grignard 1, Step 1) and imidazole (1.5 eq.) was added chlorotriisopropylsilane (1.2 eq.). The resulting solution was allowed to stir at RT for 24 h before it was diluted with hexanes and washed with water and brine. The organic extract was dried over Na₂SO₄, filtered and the filtrate concentrated in vacuo. Purification of the crude product thus obtained by way of column chromatography (SiO₂, Hex→7:3 (v/v) Hex: EtOAc) afforded the title compound as a colorless oil.

### Step 2: Grignard 3

[(2-Bromo-4,5-difluorobenzyl)oxy](triisopropyl)silane (0.2 M THF solution, 1 eq.) from the previous step was added to a 1 M THF suspension of Rieke magnesium (2 eq.) at a rate as to keep the internal reaction temperature below 45° C. The resulting dark suspension was then allowed to stir at RT for 3 h before unreacted Rieke magnesium was removed via filtration. The concentration of the title compound in THF thus obtained was determined via titration to be 0.015 M.

### Example 1

### rac-(1R,3S)3′-[2′-(tert-Butoxymethyl)-3-methyl-4-biphenylyl]-5,6-Difluoro-3H-spiro[2-benzofuran-1,4′-piperidine]

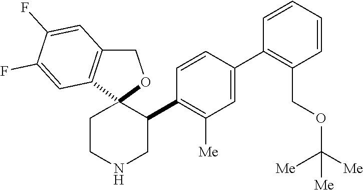

### Step 1: (4′-Bromo-3′-methyl-2-biphenylyl)methanol

To a 2:1 (v/v) toluene: ethanol solution (0.25 M) of 2,1-benzoxaborol-1(31)-ol (1 eq.) and 2-bromo-5-iodotoluene (1.2 eq.) was added was added trans-bis(triphenylphosphine) palladium(II) bromide (0.02 eq.). The vessel was repeatedly evacuated and back-filled with nitrogen. Finally, Na₂CO₃ (2 M aq. solution, 3 eq.) was added and the resulting mixture was heated at 50° C. for 12 h. The now black suspension was cooled to RT, diluted with ether and quenched with 10% aq. HCl. The aqueous layer was separated and back-extracted with ether. The combined organic extracts were then washed further with 1 N aq. NaOH, water and brine, dried over Na₂SO₄, filtered and the filtrate concentrated in vacuo. Purification of the crude product by way of flash chromatography (SiO₂, Hex→1:1 (v/v) Hex: EtOAc) afforded the title compound as a golden, yellow oil.

### Step 2: [(4′-Bromo-3′-methyl-2-biphenylyl)methoxy](triisopropyl)silane

To a DMF solution (0.24 M) of (4′-bromo-3′-methyl-2-biphenylyl)methanol (1 eq.) from the previous step and imidazole (1.5 eq.) was added chloro(triisopropyl)silane (1 eq.) dropwise at 0° C. The resulting solution was stirred at RT for 48 h before the crude reaction mixture was diluted with hexanes and washed sequentially with water, 10% aq. HCl, 1 N aq. NaOH, water and brine. The organic extract was then dried over Na₂SO₄, filtered and the filtrate concentrated in vacuo to furnish the crude title compound as a viscous oil.

### Step 3: tert-Butyl 3-(3-methyl-2′-{[(triisopropylsilyl)oxy]methyl)-4-biphenyl}-4-oxo-1-piperidinecarboxylate

Tris(dibenzylideneacetone)dipalladium(0)-chloroform adduct (0.02 eq.), 1,1′-bis(di-tert-butylphosphino)ferrocene (0.2 eq.) and sodium tert-butoxide (2.2 eq.) were combined in THF. To this suspension was then added tert-butyl 4-oxo-1-piperidinecarboxylate (1 eq., 0.23 M) and [(4′-bromo-3′-methyl-2-biphenylyl)methoxy](triisopropyl)silane (1.2 eq.) from the previous step. The resulting reaction mixture was evacuated and back-filled with N₂ before it was heated at 70° C. for 12 h. The reaction was then cooled to RT, quenched with the addition of ether and 10% aq. HCl. The aqueous layer was separated and back-extracted with ether. The combined organic extracts were washed further with water and brine, treated with activated charcoal, dried over Na₂SO₄, filtered and the filtrate concentrated in vacuo. Purification of the crude product thus obtained by way of column chromatography (SiO₂, 95:5 (v/v) Hex: EtOAc→1:1 (v/v) Hex: EtOAc) afforded the title compound as a yellow oil that solidified upon standing.

### Step 4: rac-tert-Butyl (3S,4R)-4-[2-(1,3-dioxolan-2-yl)-4,5-difluorophenyl]-4-hydroxy-3-(3-methyl-2′-{[(triisopropylsilyl)oxy]methyl}-4-biphenylyl)-1-piperidinecarboxylate

To a THF solution (0.18 M) of tert-butyl 3-(3-methyl-2′-{[(triisopropylsilyl)oxy]methyl)-4-biphenyl}-4-oxo-1-piperidinecarboxylate (1 eq.) from the previous step and anhydrous lithium chloride (2 eq.) was added Grignard 1 (2 eq.) at RT over a period of 10 min. The resulting suspension was allowed to stir at RT for another 13 h. The reaction mixture was then diluted with ether and carefully quenched with 10% aq. HCl. The aqueous layer was separated and back-extracted with ether. The combined organic extracts were washed further with brine, dried over Na₂SO₄, filtered and the filtrate concentrated in vacuo. Purification of the crude product thus obtained by way of column chromatography (SiO₂, 95:5 (v/v) Hex: EtOAc→3:7 (v/v) Hex: EtOAc) afforded the title compound as a white foam.

### Step 5: rac-tert-Butyl (1R,3′S)-5,6-difluoro-3-hydroxy-3′-(3-methyl-2′-{[(triisopropylsilyl)oxy]methyl}-4-biphenylyl)-1′H,3H-spiro[2-benzofuran-1,4′-piperidine]-1′-carboxylate

To a 5:1 (v/v) acetone: water solution (0.044 M) of rac-tert-butyl (3S,4R)-4-[2-(1,3-dioxolan-2-yl)-4,5-difluorophenyl]-4-hydroxy-3-(3-methyl-2′-{[(triisopropylsilyl)oxy]methyl}-4-biphenylyl)-1-piperidinecarboxylate (1 eq.) from the previous step was added PPTS (0.1 eq.). The resulting solution was heated at 80° C. for 4 h. The volatiles were then removed in vacuo and the resulting residue was partitioned between ether and 1 N aq. NaOH. The aqueous wash was separated and back-extracted with ether. The combined organic extracts were washed further with water and brine, dried over Na₂SO₄ and filtered. Concentration of the filtrate thus obtained afforded the crude title compound as a white solid.

### Step 6: rac-tert-Butyl (3S,4R)-4-[4,5-difluoro-2-(hydroxymethyl)phenyl]-4-hydroxy-3-(3-methyl-2′-{[(triisopropylsilyl)oxy]methyl}-4-biphenylyl)-1-piperidinecarboxylate

To a THF solution (0.11 M) of rac-tert-butyl (1R,3′S)-5,6-difluoro-3-hydroxy-3′-(3-methyl-2′-{[(triisopropylsilyl)oxy]methyl}-4-biphenylyl)-1′H,3H-spiro[2-benzofuran-1,4′-piperidine]-1′-carboxylate (1 eq.) from the previous step was added lithium borohydride (1.9 eq.). After stirring at RT for 24 h, the reaction mixture was diluted with ether and carefully quenched with 10% aq. HCl. The organic layer was separated, washed further with 1 N aq. NaOH, water and brine, dried over Na₂SO₄, filtered and the filtrate concentrated in vacuo. Purification of the crude product thus obtained by way of column chromatography (SiO₂, 9:1 (v/v) Hex: EtOAc→EtOAc) afforded the title compound as a viscous oil.

### Step 7: rac-tert-Butyl (1R,3′S)-5,6-difluoro-3′-(3-methyl-2′-{[(triisopropylsilyl)oxy]methyl}-4-biphenylyl)-1′H,3H-spiro[2-benzofuran-1,4′-piperidine]-1′-carboxylate

To a THF solution (0.05 M) of rac-tert-butyl (3S,4R)-4-[4,5-difluoro-2-(hydroxymethyl)phenyl]-4-hydroxy-3-(3-methyl-2′-{[(triisopropylsilyl)oxy]methyl}-4-biphenylyl)-1-piperidinecarboxylate (1 eq.) from the previous step and triethylamine (3 eq.) was added at 0 methanesulfonyl chloride (1.5 eq.). The resulting suspension was allowed to warm to RT over 14 h. The volatiles were then removed in vacuo and the residue thus obtained was partitioned between ether and 1 N aq. NaOH. The aqueous layer was separated and back-extracted with ether. The combined organic extracts were washed further with water and brine, dried over Na₂SO₄, filtered and the filtrate concentrated in vacuo. Purification of the crude product thus obtained by way of column chromatography (SiO₂, 9:1 (v/v) Hex: EtOAc→EtOAc) afforded the title compound as a white foam.

### Step 8: rac-tert-Butyl (1R,3′S)-5,6-difluoro-3′-[2′-(hydroxymethyl)-3-methyl-4-biphenylyl]-1′H,3H-spiro[2-benzofuran-1,4′-piperidine]-1′-carboxylate

To a THF solution (0.1 M) of rac-tert-butyl (1R,3′S)-5,6-difluoro-3′-(3-methyl-2′-{[(triisopropylsilyl)oxy]methyl}-4-biphenylyl)-1′H,3H-spiro[2-benzofuran-1,4′-piperidine]-1′-carboxylate (1 eq.) from the previous step was added TBAF (1 M THF solution, 1.5 eq.). After stirring at RT for 3 h, the volatiles were then removed in vacuo and the resulting residue was partitioned between ether and water. The organic layer was separated, washed further with 1 N aq. NaOH, 10% aq. HCl and water, dried over Na₂SO₄, filtered and the filtrate concentrated in vacuo. Purification of the crude product thus obtained by way of column chromatography (SiO₂, 9:1 (v/v) Hex: EtOAc→EtOAc) afforded the title compound as a white solid.

### Step 9: rac-(1R,3′5)3′-[2′-(tert-Butoxymethyl)-3-methyl-4-biphenylyl]-5,6-Difluoro-3H-spiro[2-benzofuran-1,4′-piperidine]

To a CH₂Cl₂ solution (0.05 M) of rac-tert-butyl (1R,3′S)-5,6-difluoro-3′-[2′-(hydroxymethyl)-3-methyl-4-biphenylyl]-1′H,3H-spiro[2-benzofuran-1,4′-piperidine]-1′-carboxylate (1 eq.) from the previous step was added HCl (4.0 M dioxane solution, 30 eq.). The resulting solution was stirred at RT for 3 h. Following the removal of the volatiles in vacuo, the resulting residue was partitioned between EtOAc and 1 N aq. NaOH. The organic layer was separated, washed further with water and brine, dried over Na₂SO₄, filtered and the filtrate concentrated in vacuo. Purification of the crude product thus obtained by column chromatography (SiO₂, 95:5 (v/v) CH₂Cl₂: 2.0 M NH₃ in MeOH) afforded the title compound as a viscous oil. MS (ESI+, M+H): 478.3.

### Example 2

### rac-{4′-[(1R,3′S)-5,6-Difluoro-3H-spiro[2-benzofuran-1,4′-piperidin]-3′-yl]-3′-methyl-2-biphenylyl}methanol

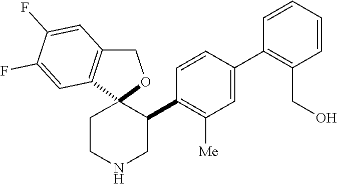

To a CH₂Cl₂ solution (0.02 M) of rac-tert-butyl (1R,3′S)-5,6-difluoro-3′-[2′-(hydroxymethyl)-3-methyl-4-biphenylyl]-1′H,3H-spiro[2-benzofuran-1,4′-piperidine]-1′-carboxylate (1 eq., Example 1, Step 8) was added zinc bromide (10 eq.). The resulting suspension was sonicated for 10 min and then stirred at RT for 16 h. The reaction was then quenched with 1 N aq. NaOH and extracted with EtOAc. The combined organic extracts were washed further with 1 N aq. NaOH, water and brine, dried over Na₂SO₄, filtered and the filtrate concentrated in vacuo. Purification of the crude product thus obtained by column chromatography (SiO₂, 95:5 (v/v) CH₂Cl₂: 2.0 M NH₃ in MeOH) afforded the title compound as a white solid. MS (ESI+, M+H): 422.3.

### Example 3

### rac-{4′-[(1R,3′S)-5,6-Difluoro-3H-spiro[2-benzofuran-1,4′-piperidin]-3′-yl]-3′-methyl-2-biphenylyl}acetonitrile

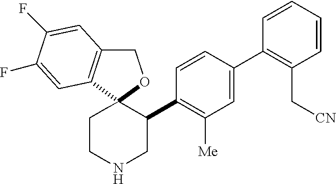

### Step 1: rac-tert-Butyl (1R,3′S)-5,6-difluoro-3′-(3-methyl-2′-{[methylsulfonyl]oxy}methyl)-4-biphenylyl)-1′H,3H-spiro[2-benzofuran-1,4′-piperidine]-1′-carboxylate

To a THF solution (0.04 M) of rac-tert-butyl (1R,3′S)-5,6-difluoro-3′-[2′-(hydroxymethyl)-3-methyl-4-biphenylyl]-1′H,3H-spiro[2-benzofuran-1,4′-piperidine]-1′-carboxylate (1 eq., Example 1, Step 8) and triethylamine (3 eq.) was added at 0° C. methanesulfonyl chloride (1.5 eq.). The resulting suspension was allowed to warm to RT over 3 h. The reaction was quenched with water and extracted with ether. The combined organic extracts were then washed further with 1 N aq. NaOH, water and brine, dried over Na₂SO₄ and filtered. Concentration of the filtrate in vacuo afforded the title compound as a white foam.

### Step 2: rac-tert-Butyl (1R,3′S)-3′-[2′-(cyanomethyl)-3-methyl-4-biphenylyl]-5,6-difluoro-1′H,3H-spiro[2-benzofuran-1,4′-piperidine]-1′-carboxylate

To a DMF solution (0.1 M) of rac-tert-butyl (1R,3′S)-5,6-difluoro-3′-(3-methyl-2′-{[methylsulfonyl]oxy}methyl)-4-biphenylyl)-1′H,3H-spiro[2-benzofuran-1,4′-piperidine]-1′-carboxylate (1 eq.) from the previous step was added potassium cyanide (1.5 eq.) and tetrabutylammonium iodide (0.1 eq.). The resulting solution was then stirred at RT for 48 h. The reaction was then diluted with water and extracted with ether. The combined organic extracts were washed further with water and brine, dried over MgSO₄, filtered and the filtrate concentrated in vacuo. Purification of the crude product thus obtained by column chromatography (SiO₂, 9:1 (v/v) Hex: EtOAc→3:7 (v/v) Hex: EtOAc) afforded the title compound as a white solid.

### Step 3: rac-{4′-[(1R,3′S)-5,6-Difluoro-3H-spiro[2-benzofuran-1,4′-piperidin]-3′-yl]-3′-methyl-2-biphenylyl}acetonitrile

To a CH₂Cl₂ solution (0.013 M) of rac-tert-butyl (1R,3′5)-3′-[2′-(cyanomethyl)-3-methyl-4-biphenylyl]-5,6-difluoro-1′H,3H-spiro[2-benzofuran-1,4′-piperidine]-1′-carboxylate (1 eq.) from the previous step was added zinc bromide (10 eq.). The resulting suspension was sonicated for 10 min and then stirred at RT for 16 h. The reaction was then quenched with 1 N aq. NaOH and extracted with EtOAc. The combined organic extracts were washed further with 1 N aq. NaOH, water and brine, dried over Na₂SO₄, filtered and the filtrate concentrated in vacuo. Purification of the crude product thus obtained by column chromatography (SiO₂, 95:5 (v/v) CH₂Cl₂: 2.0 M NH₃ in MeOH) afforded the title compound as a white solid. MS (ESI+, M+H): 431.3.

### Example 4

### rac-N-(2-{4′-[(1R,3′S)-5,6-Difluoro-3H-spiro[2-benzofuran-1,4′-piperidin]-3′-yl]-3′-methyl-2-biphenylyl}ethyl)acetamide

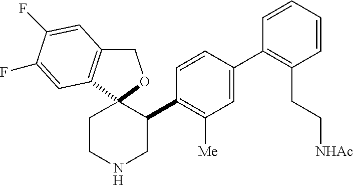

### Step 1: rac-tert-Butyl (1R,3′S)-3′-{2′-[2-(acetylamino)ethyl]-3-methyl-4-biphenylyl}-5,6-difluoro-1′H,3H-spiro[2-benzofuran-1,4′-piperidine]-1′-carboxylate

To a methanol solution (0.05 M) of rac-tert-butyl (1R,3′S)-3′-[2′-(cyanomethyl)-3-methyl-4-biphenylyl]-5,6-difluoro-1′H,3H-spiro[2-benzofuran-1,4′-piperidine]-1′-carboxylate (1 eq., Example 3, Step 2) and cobalt(III) chloride hexahydrate (2 eq.) was added sodium borohydride (10 eq.) at 0° C. The reaction solution was allowed to warm to RT over 4 h before it was diluted with dichloromethane and quenched with 1 N aq. NaOH. The resulting emulsion was then filtered through a pad of celite and the insolubles were rinsed with dichloromethane. The filtrate was washed further with brine, dried over Na₂SO₄ and filtered again. Removal of the volatiles in vacuo then afforded a yellow foam. The crude amine was immediately taken up in dichloromethane (0.05 M) and then added Hunig's base (1.5 eq.) and acetyl chloride (1.2 eq.). The resulting solution was stirred at RT for 16 h. The reaction mixture was then diluted with ether and washed sequentially with 10% aq. HCl, 1 N aq. NaOH, water and brine. The organic extract thus obtained was dried over Na₂SO₄, filtered and the filtrate concentrated in vacuo. Further purification by way of column chromatography (SiO₂, 4:1 (v/v) Hex: EtOAc→EtOAc) afforded the title compound as a white solid.

### Step 2: rac-N-(2-{4′-[(1R,3′S)-5,6-Difluoro-3H-spiro[2-benzofuran-1,4′-piperidin]-3′-yl]-3′-methyl-2-biphenylyl}ethyl)acetamide

To a CH₂Cl₂ solution (0.05 M) of rac-tert-butyl (1R,3′S)-3′-{2′-[2-(acetylamino)ethyl]-3-methyl-4-biphenylyl}-5,6-difluoro-1′H,3H-spiro[2-benzofuran-1,4′-piperidine]-1′-carboxylate (1 eq.) from the previous step was added HCl (4.0 M dioxane solution, 30 eq.). The resulting solution was stirred at RT for 3 h. Following the removal of the volatiles in vacuo, the resulting residue was partitioned between EtOAc and 1 N aq. NaOH. The organic layer was separated, washed further with water and brine, dried over Na₂SO₄, filtered and the filtrate concentrated in vacuo. Purification of the crude product thus obtained by column chromatography (SiO₂, 94:6 (v/v) CH₂Cl₂: 2.0 M NH₃ in MeOH) afforded the title compound as a white foam. MS (ESI+, M+H): 477.3. ¹H NMR δ (ppm, CDCl₃): 7.67 (1H, d, J=8.0 Hz), 7.31-7.22 (2H, m), 7.15 (1 H, d, J=7.5 Hz), 7.06-6.97 (2H, m), 6.86 (1H, s), 6.79 (1H, t, J=8.2 Hz), 5.15 (1H, s), 4.96 (1H, d, J=12.3 Hz), 4.81 (1H, d, J=12.3 Hz), 3.45 (1H, d, J=11.9 Hz), 3.37 (1H, dd, J=11.9, 3.7 Hz), 3.31-3.13 (4H, m), 3.05 (1H, dd, J=11.6, 3.7 Hz), 2.76-2.67 (2H, m), 2.21 (3H, s), 2.11-1.97 (2H, m), 1.93-1.80 (4H, m). Human Renin IC₅₀ (buffer): 0.2 nM. Human Renin IC₅₀ (plasma): 1.4 nM.

### Example 5

### rac-(1R,3′S)-3′-[2′-(3-Methoxypropyl)-3-methyl-4-biphenylyl]-5-methyl-3,5-dihydro-6H-spiro[furo[3,4-c]pyridine-1,4′-piperidin]-6-one

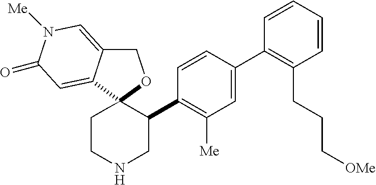

### Step 1: rac-tert-Butyl (3S,4R)-4-hydroxy-4-(2-methoxy-5-{[(triisopropylsilyl)oxy]methyl}-4-pyridinyl)-3-(3-methyl-2′-{[(triisopropylsilyl)oxy]methyl}-4-biphenylyl)-1-piperidinecarboxylate

To a THF solution (0.08 M) of tert-butyl 3-(3-methyl-2′-{[(triisopropylsilyl)oxy]methyl)-4-biphenyl}-4-oxo-1-piperidinecarboxylate (1 eq., Example 1, Step 3) and anhydrous lithium chloride (2 eq.) was added Grignard 2 (1.7 eq.) at RT over a period of 10 min. The resulting solution was allowed to stir at RT for another 8 h. The reaction mixture was then diluted with ether and carefully quenched with 10% aq. HCl. The aqueous layer was separated and back-extracted with ether. The combined organic extracts were washed further with 1 N aq. NaOH, water and brine, dried over Na₂SO₄, filtered and the filtrate concentrated in vacuo. Purification of the crude product thus obtained by way of column chromatography (SiO₂, 95:5 (v/v) Hex: EtOAc→3:7 (v/v) Hex: EtOAc) afforded the title compound as a pale yellow foam.

### Step 2: rac-tert-Butyl (3S,4R)-4-hydroxy-4-[5-(hydroxymethyl}-2-methoxy-4-pyridinyl)]-3-[2′-(hydroxymethyl)-3-methyl-4-biphenylyl)-1-piperidinecarboxylate

To a THF solution (0.1 M) of rac-tert-butyl (3S,4R)-4-hydroxy-4-(2-methoxy-5-{[(triisopropylsilyl)oxy]methyl}-4-pyridinyl)-3-(3-methyl-2′-{[(triisopropylsilyl)oxy]methyl}-4-biphenylyl)-1-piperidinecarboxylate (1 eq.) from the previous step was added TBAF (1 M THF solution, 3 eq.). After stirring at RT for 16 h, the volatiles were then removed in vacuo and the resulting residue was partitioned between ether and water. The organic layer was separated, washed further with 1 N aq. NaOH, 10% aq. HCl, water and brine, dried over Na₂SO₄, filtered and the filtrate concentrated in vacuo. Purification of the crude product thus obtained by way of column chromatography (SiO₂, 7:3 (v/v) Hex: EtOAc→EtOAc) afforded the title compound as a white solid.

### Step 3: rac-tert-Butyl (1R,3′S)-6-methoxy-3′-(3-methyl-2′-{[(methylsulfonyl)oxy]methyl}-4-biphenylyl)-1′H,3H-spiro[faro[3,4-c]pyridine-1,4′-piperidine]-1′-carboxylate

To a THF solution (0.074 M) of rac-tert-butyl (3S,4R)-4-hydroxy-4-[5-(hydroxymethyl}-2-methoxy-4-pyridinyl)]-3-[2′-(hydroxymethyl)-3-methyl-4-biphenylyl)-1-piperidinecarboxylate (1 eq.) from the previous step and triethylamine (5.8 eq.) was added at 0 methanesulfonyl chloride (3 eq.). The resulting suspension was allowed to warm to RT over 18 h. The reaction was quenched with water and extracted with ether. The combined organic extracts were then washed further with 1 N aq. NaOH, water and brine, dried over Na₂SO₄ and filtered. Concentration of the filtrate in vacuo afforded the title compound as a white foam.

### Step 4: rac-tert-Butyl (1R,3′S)-3′-[2′-(iodomethyl)-3-methyl-4-biphenylyl]-6-methoxy-1′H,3H-spiro[furo[3,4-c]pyridine-1,4′-piperidine]-1′-carboxylate

To an acetone solution (0.08 M) of rac-tert-butyl (1R,3′S)-6-methoxy-3′-(3-methyl-2′-{[(methylsulfonyl)oxy]methyl}-4-biphenylyl)-1′H,3H-spiro[furo[3,4-c]pyridine-1,4′-piperidine]-1′-carboxylate (1 eq.) from the previous step was added sodium iodide (10 eq.). The resulting solution was heated at reflux for 5 h before the volatiles were removed in vacuo and the resulting residue partitioned between ether and water. The organic layer was separated, dried over Na₂SO₄ and filtered. Concentration of the filtrate in vacuo afforded the title compound as a pale yellow foam.

### Step 5: rac-tert-Butyl (1R,3′S)-3′-[2′-(3-tert-butoxy-3-oxopropyl)-3-methyl-4-biphenylyl]-6-methoxy-1′H,3H-spiro[furo[3,4-c]pyridine-1,4′-piperidine]-1′-carboxylate

To a THF solution (0.08 M) of diisopropylamine (1.3 eq.) was added dropwise n-butyl lithium (1.6 M hexane solution, 1.2 eq.) at −78° C. The resulting solution was stirred at −78° C. for 15 min before neat tert-butyl acetate (1 eq.) was added dropwise over 10 min. The resulting mixture was stirred at −78° C. for another 30 min before rac-tert-butyl (1R,3′S)-3′-[2′-(iodomethyl)-3-methyl-4-biphenylyl]-6-methoxy-1′H,3H-spiro[furo[3,4-c]pyridine-1,4′-piperidine]-1′-carboxylate (1 eq.) from the previous step was added as a 0.2 M THF solution dropwise over 10 min. The reaction mixture thus obtained was stirred at −78° C. for 30 min and then warmed to 0° C. over 1 h. Finally, the reaction was quenched with sat. aq. NH₄Cl and extracted with ether. The combined organic extracts were washed further with water and brine, dried over Na₂SO₄, filtered and the filtrate concentrated in vacuo. Purification of the crude product thus obtained by way of column chromatography (SiO₂, 7:3 (v/v) Hex: EtOAc→EtOAc) afforded the title compound as a white foam.

### Step 6: rac-tert-Butyl (1R,3′S)-3′42′-(3-hydroxypropyl)-3-methyl-4-biphenylyl]-6-methoxy-1′H,3H-spiro[furo[3,4-c]pyridine-1,4′-piperidine]-1′-carboxylate

To a dichloromethane solution (0.066 M) of rac-tert-butyl (1R,3′S)-3′-[2′-(3-tert-butoxy-3-oxopropyl)-3-methyl-4-biphenylyl]-6-methoxy-1′H,3H-spiro[furo[3,4-c]pyridine-1,4′-piperidine]-1′-carboxylate (1 eq.) from the previous step was added diisobutylaluminum hydride (1.5 M toluene solution, 4 eq.) dropwise at 0° C. The resulting mixture was stirred at RT for 16 h before it was quenched with water and extracted with EtOAc. The combined organic extracts were then washed further with 1 N aq. NaOH, water and brine, dried over Na₂SO₄, filtered and the filtrate concentrated in vacuo. Purification of the crude product thus obtained by way of column chromatography (SiO₂, 4:1 (v/v) Hex: EtOAc→EtOAc) afforded the title compound as a white foam.

### Step 7: rac-tert-Butyl (1R,3′S)-3′-[2′-(3-methoxypropyl)-3-methyl-4-biphenylyl]-5-methyl-6-oxo-5,6-dihydro-1′H,3H-spiro[furo[3,4-c]pyridine-1,4′-piperidine]-1′-carboxylate

To a DMF solution (0.092 M) of rac-tert-butyl (1R,3′S)-3′42′-(3-hydroxypropyl)-3-methyl-4-biphenylyl]-6-methoxy-1′H,3H-spiro[furo[3,4-c]pyridine-1,4′-piperidine]-1′-carboxylate (1 eq.) from the previous step was added iodomethane (10 eq.) and sodium hydride (60% w/w dispersion in paraffin oil, 3 eq.). The resulting mixture was then heated at 45° C. for six days. The reaction was quenched with water and extracted with EtOAc. The combined organic extracts were washed further with 10% aq. HCl, 1 N aq. NaOH, water and brine, dried over Na₂SO₄, filtered and the filtrate concentrated in vacuo. Purification of the crude product thus obtained by way of column chromatography (SiO₂, 1:1 (v/v) Hex: EtOAc→EtOAc) afforded the title compound as a white foam.

### Step 8: rac-(1R,3′S)-3′-[2′-(3-Methoxypropyl)-3-methyl-4-biphenylyl]-5-methyl-3,5-dihydro-6H-spiro[furo[3,4-c]pyridine-1,4′-piperidin]-6-one

To a CH₂Cl₂ solution (0.05 M) of rac-tert-butyl (1R,3′S)-3′-[2′-(3-methoxypropyl)-3-methyl-4-biphenylyl]-5-methyl-6-oxo-5,6-dihydro-1′H,3H-spiro[furo[3,4-c]pyridine-1,4′-piperidine]-1′-carboxylate (1 eq.) from the previous step was added HCl (4.0 M dioxane solution, 30 eq.). The resulting solution was stirred at RT for 3 h. Following the removal of the volatiles in vacuo, the crude product thus obtained was directly subjected to purification by way of column chromatography (SiO₂, 94:6 (v/v) CH₂Cl₂: 2.0 M NH₃ in MeOH) to afford the title compound as a white foam. MS (ESI+, M+H): 459.3. ¹H NMR δ (ppm, CDCl₃): 7.62 (1H, d, J=8.0 Hz), 7.29-7.18 (4 H, m), 7.15 (1H, d, J=7.5 Hz), 7.03 (1H, d, J=8.1 Hz), 6.95 (2H, d, J=5.2 Hz), 6.45 (1H, s), 4.84 (1H, d, J=11.4 Hz), 4.73 (1H, d, J=11.4 Hz), 3.46 (3H, s), 3.40 (2H, t, J=11.3 Hz), 3.29-3.13 (5H, m), 3.04 (1H, d, J=9.4 Hz), 2.60 (2H, td, J=7.7, 4.6 Hz), 2.31 (3H, s), 1.98 (1H, td, J=12.7, 4.6 Hz), 1.88 (1H, d, J=13.7 Hz), 1.75 (1H, s), 1.68-1.53 (3H, m). Human Renin IC₅₀ (buffer): 1.6 nM. Human Renin IC₅₀ (plasma): 18 nM

### Example 6

### rac-(1R,3′S)-5-methyl-3′-[3-methyl-2′-(3-oxobutyl)-4-biphenylyl]-3,5-dihydro-6H-spiro[furo[3,4-c]pyridine-1,4′-piperidin]-6-one

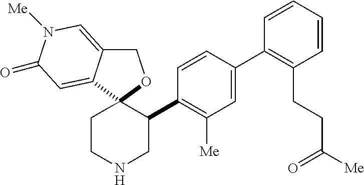

### Step 1: rac-tert-Butyl (1R,3′S)-3′-[2′-(5-tert-butoxy-3,5-dioxopentyl)-3-methyl-4-biphenylyl]-6-methoxy-1′H,3H-spiro[furo[3,4-c]pyridine-1,4′-piperidine]-1′-carboxylate

To a THF solution (0.07 M) of diisopropylamine (2.3 eq.) was added dropwise n-butyl lithium (1.6 M hexane solution, 2.2 eq.) at −78° C. The resulting solution was stirred at −78° C. for 15 min before neat tert-butyl acetate (2 eq.) was added dropwise over 10 min. The resulting mixture was stirred at −78° C. for another 30 min before rac-tert-butyl (1R,3′S)-3′-[2′-(iodomethyl)-3-methyl-4-biphenylyl]-6-methoxy-1′H,3H-spiro[furo[3,4-c]pyridine-1,4′-piperidine]-1′-carboxylate (1 eq., Example 5, Step 4) was added as a 0.2 M THF solution dropwise over 10 min. The reaction mixture thus obtained was stirred at −78° C. for 30 min and then warmed to RT over 48 h. Finally, the reaction was quenched with sat. aq. NH₄Cl and extracted with ether. The combined organic extracts were washed further with water and brine, dried over Na₂SO₄, filtered and the filtrate concentrated in vacuo. Purification of the crude product thus obtained by way of column chromatography (SiO₂, 7:3 (v/v) Hex: EtOAc→EtOAc) afforded the title compound as a white foam.

### Step 2: rac-tert-Butyl (1R,3′S)-3′-[2′-(5-tert-butoxy-3,5-dioxopentyl)-3-methyl-4-biphenylyl]-5-methyl-6-oxo-5,6-dihydro-1′H,3H-spiro[furo[3,4-c]pyridine-1,4′-piperidine]-1′-carboxylate

To an acetonitrile solution (0.097 M) of rac-tert-butyl (1R,3′ S)-3′-[2′-(5-tert-butoxy-3,5-dioxopentyl)-3-methyl-4-biphenylyl]-6-methoxy-1′H,3H-spiro[furo[3,4-c]pyridine-1,4′-piperidine]-1′-carboxylate (1 eq.) from the previous step was added iodomethane (5 eq.) and sodium iodide (3 eq.). The resulting mixture was then heated at 45° C. for six days. After the removal of the volatiles in vacuo, the resulting residue was diluted with water and extracted with EtOAc. The combined organic extracts were washed further with 10% aq. HCl, 1 N aq. NaOH, water and brine, dried over Na₂SO₄, filtered and the filtrate concentrated in vacuo. Purification of the crude product thus obtained by way of column chromatography (SiO₂, 1:1 (v/v) Hex: EtOAc→EtOAc) afforded the title compound as a white foam.

### Step 3: rac-(1R,3′S)-5-methyl-3′-[3-methyl-2′-(3-oxobutyl)-4-biphenylyl]-3,5-dihydro-6H-spiro[furo[3,4-c]pyridine-1,4′-piperidin]-6-one

To a dichloroethane solution (0.03 M) of rac-tert-butyl (1R,3′ S)-3′-[2′-(5-tert-butoxy-3,5-dioxopentyl)-3-methyl-4-biphenylyl]-5-methyl-6-oxo-5,6-dihydro-1′H,3H-spiro[furo[3,4-c]pyridine-1,4′-piperidine]-1′-carboxylate (1 eq.) from the previous step was added HCl (4.0 M dioxane solution, 30 eq.). The resulting solution was heated a reflux for 3 days. Following the removal of the volatiles in vacuo, the crude product thus obtained was directly subjected to purification by way of column chromatography (SiO₂, 94:6 (v/v) CH₂Cl₂: 2.0 M NH₃ in MeOH) to afford the title compound as a white foam. MS (ESI+, M+H): 457.3.

### Example 7

### rac-N-(2-{4′-[(1R,3′S)-5,6-Difluoro-3H-spiro[2-benzofuran-1,4′-piperidin]-3′-yl]-3′,6-dimethyl-2-biphenylyl}ethyl)acetamide

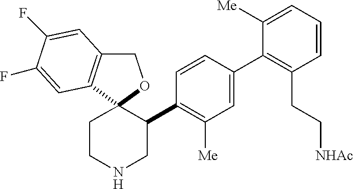

### Step 1: Methyl 3-methyl-2-(4,4,5,5-tetramethyl-1,3,2-dioxaborolan-2-yl)benzoate

To a dioxane solution (0.05 M) of bis(pinacolato)diborane (1.4 eq.), methyl 2-bromo-3-methylbenzoate (1 eq.) and potassium acetate (3 eq.) was added [1,1′-bis(diphenylphosphino)ferrocene]dichloropalladium(II) (0.05 eq.). The vessel was evacuated and back-filled three times with nitrogen before the reaction mixture was heated at 90° C. for 14 h. The now black suspension was cooled to RT, quenched with sat. aq. NH₄Cl and extracted with ether. The combined organic extracts were washed further with water and brine, treated with activated charcoal, dried over Na₂SO₄, filtered through a pad of celite and the filtrate concentrated in vacuo. Purification of the crude product thus obtained by way of column chromatography (SiO₂, Hex→1:1 (v/v) Hex: EtOAc) afforded the title compound as a lime green solid.

### Step 2: Methyl 4′-bromo-3′,6-dimethyl-2-biphenylcarboxylate

To a 2:1 (v/v) toluene: ethanol solution (0.034 M) of methyl 3-methyl-2-(4,4,5,5-tetramethyl-1,3,2-dioxaborolan-2-yl)benzoate (1 eq.) from the previous step and 2-bromo-5-iodotoluene (1.2 eq.) was added was added trans-bis(triphenylphosphine) palladium(II) bromide (0.12 eq.). The vessel was repeatedly evacuated and back-filled with nitrogen. Finally, Na₂CO₃ (2 M aq. solution, 3 eq.) was added and the resulting mixture was heated at 50° C. for 12 h. The now black suspension was cooled to RT, diluted with ether and quenched with 10% aq. HCl. The aqueous layer was separated and back-extracted with ether. The combined organic extracts were then washed further with 1 N aq. NaOH, water and brine, dried over Na₂SO₄, filtered and the filtrate concentrated in vacuo. Purification of the crude product by way of flash chromatography (SiO₂, Hex→1:1 (v/v) Hex: EtOAc) afforded the title compound as a colorless oil.

### Step 3: (4′-Bromo-3′,6-dimethyl-2-biphenylyl)methanol

To a dichloromethane solution (0.078 M) of methyl 4′-bromo-3′,6-dimethyl-2-biphenylcarboxylate (1 eq.) from the previous step was added diisobutylaluminum hydride (1.5 M toluene solution, 2.2 eq.) dropwise at −78° C. The resulting mixture was then allowed to warm slowly to RT over 16 h before it was quenched with 10% aq. HCl and extracted with ether. The combined organic extracts were then washed further with 1 N aq. NaOH, water and brine, dried over MgSO₄ and filtered. Concentration of the filtrate in vacuo afforded the title compound as a viscous oil.

### Step 4: [(4′-Bromo-3′,6-dimethyl-2-biphenylyl)methoxy](tert-butyl)dimethylsilane

To a DMF solution (0.17 M) of (4′-bromo-3′,6-dimethyl-2-biphenylyl)methanol (1 eq.) from the previous step and imidazole (1.5 eq.) was added tert-butyl(chloro)dimethylsilane (1 eq.) at 0° C. The resulting solution was stirred at RT for 18 h before the crude reaction mixture was diluted with hexanes and washed sequentially with water, 10% aq. HCl, 1 N aq. NaOH, water and brine. The organic extract was then dried over Na₂SO₄, filtered and the filtrate concentrated in vacuo. Purification of the crude product by way of flash chromatography (SiO₂, Hex→3:1 (v/v) Hex: EtOAc) afforded the title compound as a colorless oil.

### Step 5: tert-Butyl 3-[2′-({[tert-butyl(dimethyl)silyl]oxy}methyl)-3,6′-dimethyl-4-biphenyl}-4-oxo-1-piperidinecarboxylate

Tris(dibenzylideneacetone)dipalladium(0)-chloroform adduct (0.02 eq.), 1,1′-bis(di-tert-butylphosphino)ferrocene (0.2 eq.) and sodium tert-butoxide (2.2 eq.) were combined in THF. To this suspension was then added tert-butyl 4-oxo-1-piperidinecarboxylate (1 eq., 0.23 M) and [(4′-bromo-3′,6-dimethyl-2-biphenylyl)methoxy](tert-butyl)dimethylsilane (1.2 eq.) from the previous step. The resulting reaction mixture was evacuated and back-filled with N₂ before it was heated at 70° C. for 12 h. The reaction was then cooled to RT, quenched with the addition of ether and 10% aq. HCl. The aqueous layer was separated and back-extracted with ether. The combined organic extracts were washed further with water and brine, treated with activated charcoal, dried over Na₂SO₄, filtered and the filtrate concentrated in vacuo. Purification of the crude product thus obtained by way of column chromatography (SiO₂, 95:5 (v/v) Hex:EtOAc→1:1 (v/v) Hex: EtOAc) afforded the title compound as a yellow oil.

### Step 6: rac-tert-Butyl (3S,4R)-3-[2′-({[tert-butyl(dimethyl)silyl]oxy}methyl)-3,6′-dimethyl-4-biphenylyl]-4-(4,5-difluoro-2-{[(triisopropylsilyl)oxy]methyl}phenyl)-4-hydroxy-1-piperidinecarboxylate

To a THF solution (0.18 M) of tert-butyl 3-[2′-({[tert-butyl(dimethyl)silyl]oxy}methyl)-3,6′-dimethyl-4-biphenyl}-4-oxo-1-piperidinecarboxylate (1 eq.) from the previous step and anhydrous lithium chloride (2 eq.) was added Grignard 3 (5 eq.) at RT over a period of 10 min. The resulting suspension was allowed to stir at RT for another 12 h. The reaction mixture was then diluted with ether and carefully quenched with 10% aq. HCl. The aqueous layer was separated and back-extracted with ether. The combined organic extracts were washed further with 1 N aq. NaOH, water and brine, dried over Na₂SO₄, filtered and the filtrate concentrated in vacuo. Purification of the crude product thus obtained by way of column chromatography (SiO₂, 95:5 (v/v) Hex: EtOAc→3:7 (v/v) Hex: EtOAc) afforded the title compound as a 1:1 mixture of atropisomers.

### Step 7: rac-tert-Butyl (3S,4R)-4-[4,5-difluoro-2-(hydroxymethyl)phenyl]-4-hydroxy-3-[2′-(hydroxymethyl)-3,6′-dimethyl-4-biphenylyl]-1-piperidinecarboxylate

To a THF solution (0.045 M) of rac-tert-butyl (3S,4R)-3-[2′-({[tert-butyl(dimethyl)silyl]oxy}methyl)-3,6′-dimethyl-4-biphenylyl]-difluoro-2-{[(triisopropylsilyl)oxy]methyl}phenyl)-4-hydroxy-1-piperidinecarboxylate (1 eq.) from the previous step was added TBAF (1 M THF solution, 3 eq.). After stirring at RT for 16 h, the volatiles were then removed in vacuo and the resulting residue was partitioned between EtOAc and water. The organic layer was separated, washed further with 1 N aq. NaOH, 10% aq. HCl, water and brine, dried over Na₂SO₄, filtered and the filtrate concentrated in vacuo. Purification of the crude product thus obtained by way of column chromatography (SiO₂, 7:3 (v/v) Hex: EtOAc→EtOAc) afforded the title compound as a colorless oil.

### Step 8: rac-tert-Butyl (1R,3′S)-3′-(2′,3-dimethyl-6′-{[(methylsulfonyl)oxy]methyl}-4-biphenylyl)-5,6-difluoro-1′H,3H-spiro[2-benzofuran-1,4′-piperidine]-1′-carboxylate

To a THF solution (0.091 M) of rac-tert-butyl (3S,4R)-4-[4,5-difluoro-2-(hydroxymethyl)phenyl]-4-hydroxy-3-[2′-(hydroxymethyl)-3,6′-dimethyl-4-biphenylyl]-1-piperidinecarboxylate (1 eq.) from the previous step and triethylamine (6 eq.) was added at 0 methanesulfonyl chloride (3 eq.). The resulting suspension was allowed to warm to RT over 24 h. The reaction was quenched with water and extracted with ether. The combined organic extracts were then washed further with 1 N aq. NaOH, water and brine, dried over Na₂SO₄ and filtered. Concentration of the filtrate in vacuo afforded the title compound as a white foam.

### Step 9: rac-tert-Butyl (1R,3′S)-3′-[2′-(cyanomethyl)-3,6′-dimethyl-4-biphenylyl]-5,6-difluoro-1′H,3H-spiro[2-benzofuran-1,4′-piperidine]-1′-carboxylate

To a DMF solution (0.087 M) of rac-tert-butyl (1R,3′S)-3′-(2′,3-dimethyl-6′-{[(methylsulfonyl)oxy]methyl}-4-biphenylyl)-5,6-difluoro-1′H,3H-spiro[2-benzofuran-1,4′-piperidine]-1′-carboxylate (1 eq.) from the previous step was added potassium cyanide (1.76 eq.) and tetrabutylammonium iodide (0.3 eq.). The resulting solution was stirred at RT for 48 h. The reaction was then diluted with water and extracted with ether. The combined organic extracts were washed further with water and brine, dried over MgSO₄, filtered and the filtrate concentrated in vacuo. Purification of the crude product thus obtained by column chromatography (SiO₂, 95:5 (v/v) Hex: EtOAc→3:7 (v/v) Hex: EtOAc) afforded the title compound as a white solid.

### Step 10: rac-tert-Butyl (1R,3′S)-3′-{2′-[2-(acetylamino)ethyl]-3,6′-dimethyl-4-biphenylyl}-5,6-difluoro-1′H,3H-spiro[2-benzofuran-1,4′-piperidine]-1′-carboxylate

To a methanol solution (0.059 M) of rac-tert-butyl (1R,3′S)-3′-[2′-(cyanomethyl)-3,6′-dimethyl-4-biphenylyl]-5,6-difluoro-1′H,3H-spiro[2-benzofuran-1,4′-piperidine]-1′-carboxylate (1 eq.) from the previous step and cobalt(III) chloride hexahydrate (2 eq.) was added sodium borohydride (10 eq.) at 0° C. The reaction solution was allowed to warm to RT over 4 h before it was diluted with dichloromethane and quenched with 1 N aq. NaOH. The resulting emulsion was then filtered through a pad of celite and the insolubles were rinsed with dichloromethane. The filtrate was washed further with brine, dried over Na₂SO₄ and filtered again. Removal of the volatiles in vacuo then afforded a pale green foam. The crude amine thus obtained was immediately taken up in dichloromethane (0.059 M) and then added Hunig's base (1.5 eq.) and acetyl chloride (1.2 eq.). The resulting solution was stirred at RT for 16 h. The reaction mixture was then diluted with ether and washed sequentially with 10% aq. HCl, 1 N aq. NaOH, water and brine. The organic extract thus obtained was dried over Na₂SO₄, filtered and the filtrate concentrated in vacuo. Further purification by way of column chromatography (SiO₂, 4:1 (v/v) Hex: EtOAc→EtOAc) afforded the title compound as a 1:1 mixture of atropisomers.

### Step 11: rac-N-(2-{4′-[(1R,3′S)-5,6-Difluoro-3H-spiro[2-benzofuran-1,4′-piperidin]-3′-yl]-3′,6-dimethyl-2-biphenylyl}ethyl)acetamide

To a CH₂Cl₂ solution (0.034 M) of rac-tert-butyl (1R,3′S)-3′-{2′-[2-(acetylamino)ethyl]-3,6′-dimethyl-4-biphenylyl}-5,6-difluoro-1′H,3H-spiro[2-benzofuran-1,4′-piperidine]-1′-carboxylate (1 eq.) from the previous step was added HCl (4.0 M dioxane solution, 30 eq.). The resulting solution was stirred at RT for 2 h. Following the removal of the volatiles in vacuo, the resulting residue was directly subjected to purification by way of column chromatography (SiO₂, 94:6 (v/v) CH₂Cl₂: 2.0 M NH₃ in MeOH) to afford the title compound as a 1:1 mixture of atropisomers. MS (ESI+, M+H): 491.4.

### Example 8

### N-(2-{4′-[(1R,3′S)-5,6-Difluoro-3-oxo-3H-spiro[2-benzofuran-1,4′-piperidin]-3′-yl]-3′-methyl-2-biphenylyl}ethyl)acetamide

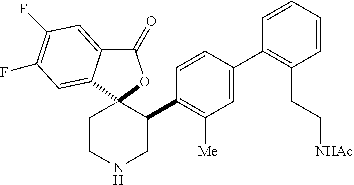

### Step 1: rac-tert-Butyl 3-(4-hydroxy-2-methylphenyl)-4-oxo-1-piperidinecarboxylate

Tris(dibenzylideneacetone)dipalladium(0)-chloroform adduct (0.02 eq.), 1,1′-bis(di-tert-butylphosphino)ferrocene (0.2 eq.) and sodium tert-butoxide (2.2 eq.) were combined in THF. To this suspension was then added tert-butyl 4-oxo-1-piperidinecarboxylate (1.5 eq.) and 4-bromo-3-methylphenyl pivalate (1 eq., 0.13 M). The resulting reaction mixture was evacuated and back-filled with N₂ before it was heated at 70° C. for 12 h. The reaction was then cooled to RT, quenched with the addition of ether and 10% aq. HCl. The aqueous layer was separated and back-extracted with ether. The combined organic extracts were washed further with water and brine, treated with activated charcoal, dried over Na₂SO₄, filtered and the filtrate concentrated in vacuo. Purification of the crude product thus obtained by way of column chromatography (SiO₂, 95:5 (v/v) Hex: EtOAc→1:1 (v/v) Hex: EtOAc) afforded the title compound as a white solid.

### Step 2: rac-tert-Butyl 3-(4-{[tert-butyl(dimethyl)silyl]oxy}-2-methylphenyl)-4-oxo-1-piperidinecarboxylate

To a DMF solution (0.16 M) of rac-tert-butyl 3-(4-hydroxy-2-methylphenyl)-4-oxo-1-piperidinecarboxylate (1 eq.) from the previous step and imidazole (1.5 eq.) was added tert-buty(chloro)dimethylsilane (1.1 eq.) and a few crystals of DMAP. The resulting solution was allowed to stir at RT for 48 h before it was diluted with ether and washed with water and brine. The organic extract was dried over Na₂SO₄, filtered and the filtrate concentrated in vacuo. Purification of the crude product thus obtained by way of column chromatography (SiO₂, Hex→1:1 (v/v) Hex: EtOAc) afforded the title compound as a colorless oil.

### Step 3: rac-tert-Butyl (1R,3′S)-3′-(4-{[tert-butyl(dimethyl)silyl]oxy}-2-methylphenyl-5,6-difluoro-3-oxo-1′H,3H-spiro[2-benzofuran-1,4′-piperidine]-1′-carboxylate

To a THF solution (0.09 M) of 2-bromo-4,5-difluorobenzoic acid (3 eq.) was added at, −30° C., di-n-butyl magnesium (1 M THF solution, 3 eq.) dropwise over 5 min. After 15 min of stirring at −30° C., the reaction mixture was cooled further to −78° C. before n-butyl lithium (1.6 M hexane solution, 3 eq.) was added dropwise over 5 min. After 30 min of stirring at −78° C., cerium trichloride (3 eq.) was added in one rapid portion and the resulting suspension was warmed to 0° C. over 30 min. Finally, rac-tert-butyl 3-(4-{[tert-butyl(dimethyl)silyl]oxy}-2-methylphenyl)-4-oxo-1-piperidinecarboxylate (1 eq.) from the previous step was added as a 0.3 M THF solution. The reaction mixture was allowed to stir at RT for 3 h before it was carefully quenched with 10% HCl. The aqueous layer was separated and back-extracted with ether. The combined organic extracts were washed further with water and brine, dried over Na₂SO₄, filtered and the filtrate concentrated in vacuo. Purification of the crude product thus obtained by way of column chromatography (SiO₂, 98:2 (v/v) toluene: acetone→95:5 (v/v) toluene: acetone) afforded the title compound as a white solid.

### Step 4: rac-tert-Butyl (1R,3′S)-5,6-difluoro-3′-(4-hydroxy-2-methylphenyl)-3-oxo-1′H,3H-spiro[2-benzofuran-1,4′-piperidine]-1′-carboxylate

To a THF solution (0.097 M) rac-tert-butyl (1R,3′S)-3′-(4-{[tert-butyl(dimethyl)silyl]oxy}-2-methylphenyl-5,6-difluoro-3-oxo-1′H,3H-spiro[2-benzofuran-1,4′-piperidine]-1′-carboxylate (1 eq.) from the previous step was added TBAF (1 M THF solution, 1.5 eq.). After stirring at RT for 2 h, the volatiles were then removed in vacuo and the resulting residue was partitioned between ether and 10% aq. HCl. The organic layer was separated, washed further with water and brine, dried over Na₂SO₄, filtered and the filtrate concentrated in vacuo. Purification of the crude product thus obtained by way of column chromatography (SiO₂, 9:1 (v/v) Hex: EtOAc→3:7 (v/v) Hex: EtOAc) afforded the title compound as a white foam.

### Step 5: rac-tert-Butyl (1R,3′S)-5,6-difluoro-3′-(2-methyl-4-{[(trifluoromethyl)sulfonyl]oxy}phenyl)-3-oxo-1′H,3H-spiro[2-benzofuran-1,4′-piperidine]-1′-carboxylate

To a dichloromethane solution (0.05 M) rac-tert-butyl (1R,3′S)-5,6-difluoro-3′-(4-hydroxy-2-methylphenyl)-3-oxo-1′H,3H-spiro[2-benzofuran-1,4′-piperidine]-1′-carboxylate (1 eq.) from the previous step was added sequentially at 0° C. triethylamine (2.8 eq.) and trifluoromethanesulfonic anhydride (1.4 eq.). After stirring at 0° C. for 2 h, the reaction mixture was carefully quenched with sat. aq. NH₄Cl. The aqueous layer was separated and extracted further with dichloromethane. The combined organic extracts were washed with brine, dried over Na₂SO₄, filtered and the filtrate concentrated in vacuo. Purification of the crude product thus obtained by way of column chromatography (SiO₂, 9:1 (v/v) Hex: EtOAc→3:7 (v/v) Hex: EtOAc) afforded the title compound as a white foam.

### Step 6: Benzyl {2-[2-(4,4,5,5-tetramethyl-1,3,2-dioxaborolan-2-yl)phenyl]ethyl}carbamate

To a dioxane solution (0.05 M) of bis(pinacolato)diborane (1.4 eq.), benzyl [2-(2-bromophenyl)ethyl]carbamate (1 eq.) and potassium acetate (3 eq.) was added [1,1′-bis(diphenylphosphino)ferrocene]dichloropalladium(II) (0.05 eq.). The vessel was evacuated and back-filled three times with nitrogen before the reaction mixture was heated at 80° C. for 14 h. The resulting suspension was cooled to RT, quenched with sat. aq. NH₄Cl and extracted with ether. The combined organic extracts were washed further with water and brine, dried over Na₂SO₄, filtered and the filtrate concentrated in vacuo. Purification of the crude product thus obtained by way of column chromatography (SiO₂, 9:1 (v/v) Hex: EtOAc→3:7 (v/v) Hex: EtOAc) afforded the title compound as a pale yellow solid.

### Step 7: tert-Butyl (1R,3′S)-3′-[2′-(2-{[(benzyloxy)carbonyl]amino}ethyl)-3-methyl-4-biphenylyl]-5,6-difluoro-3-oxo-1′H,3H-spiro[2-benzofuran-1,4′-piperidine]-1′-carboxylate

To a THF solution (0.09 M) of rac-tert-butyl (1R,3′S)-5,6-difluoro-3′-(2-methyl-4-{[(trifluoromethyl)sulfonyl]oxy}phenyl)-3-oxo-1′H,3H-spiro[2-benzofuran-1,4′-piperidine]-1′-carboxylate (1 eq., Step 5), benzyl {2-[2-(4,4,5,5-tetramethyl-1,3,2-dioxaborolan-2-yl)phenyl]ethyl}carbamate (1 eq., Step 6), palladium(II) acetate (0.07 eq.) and 2-dicyclohexylphosphino-2′,6′-dimethoxy-1,1′-biphenyl (0.13 eq.) was added potassium phosphate (1.6 M aq. solution, 2 eq.). The resulting reaction mixture was evacuated and back-filled with N₂ before it was heated at 80° C. for 4 h. The reaction was then cooled to RT, diluted with ether and quenched with the addition of 10% aq. HCl. The aqueous layer was separated and back-extracted with ether. The combined organic extracts were washed further with water and brine, dried over Na₂SO₄, filtered and the filtrate concentrated in vacuo. Purification of the crude product thus obtained by way of column chromatography (SiO₂, 95:5 (v/v) Hex: EtOAc→3:7 (v/v) Hex: EtOAc) afforded the title compound as a racemate. Resolution of the enantiomers was then carried out on 50×500 mm Chiracel OD column with 84.7:5:5:5:0.3 (v/v/v/v) Hex: MeOH: EtOH: iPrOH: triethylamine as eluent (50 mL/min) Under this condition, the desired enantiomer exhibited a retention time of 22 min.

### Step 8: tert-Butyl (1R,3′S)-3′-[2′-(2-aminoethyl)-3-methyl-4-biphenylyl]-5,6-difluoro-3-oxo-1′H,3H-spiro[2-benzofuran-1,4′-piperidine]-1′-carboxylate

To an ethanol solution (0.01 M) of tert-butyl (1R,3′S)-3′-[2′-(2-{[(benzyloxy)carbonyl]amino}ethyl)-3-methyl-4-biphenylyl]-5,6-difluoro-3-oxo-1′H,3H-spiro[2-benzofuran-1,4′-piperidine]-1′-carboxylate (1 eq.) from the previous step was added palladium black (10% w/w on activated carbon, 0.1 eq.). The resulting suspension was evacuated and back-filled first with N₂, and then with H₂. Finally, under a balloon-maintained hydrogen atmosphere, the reaction suspension was allowed to stir at RT for 20 h. The reaction was quenched with the addition of dichloromethane and filtered through a bed of dichloromethane-wetted celite. The insolubles were washed with dichloromethane and the combined filtrate was concentrated in vacuo to furnish the title compound.

### Step 9: tert-Butyl (1R,3′S)-3′-{2′-[2-(acetylamino)ethyl]-3-methyl-4-biphenylyl}-5,6-difluoro-3-oxo-1′H,3H-spiro[2-benzofuran-1,4′-piperidine]-1′-carboxylate

To a dichloromethane solution (0.1 M) of tert-butyl (1R,3′S)-3′-[2′-(2-aminoethyl)-3-methyl-4-biphenylyl]-5,6-difluoro-3-oxo-1′H,3H-spiro[2-benzofuran-1,4′-piperidine]-1′-carboxylate (1 eq.) from the previous step was added triethylamine (2 eq.) followed by acetyl chloride (1.2 eq.). The resulting reaction mixture was stirred at RT for 16 h. The volatiles were then removed in vacuo and the crude product thus obtained was directly subjected to purification by way of column chromatography (SiO₂, 4:1 (v/v) Hex: EtOAc→EtOAc) to furnish the title compound.

### Step 10: N-(2-{4′[(1R,3′S)-5,6-Difluoro-3-oxo-3H-spiro[2-benzofuran-1,4′-piperidin]-3′-yl]-3′-methyl-2-biphenylyl}ethyl)acetamide

To a CH₂Cl₂ solution (0.058 M) of tert-butyl (1R,3′S)-3′-{2′-[2-(acetylamino)ethyl]-3-methyl-4-biphenylyl}-5,6-difluoro-3-oxo-1′H,3H-spiro[2-benzofuran-1,4′-piperidine]-1′-carboxylate (1 eq.) from the previous step was added HCl (4.0 M dioxane solution, 30 eq.). The resulting solution was stirred at RT for 1.5 h. The volatiles were then removed in vacuo to afford the title compound as an HCl salt. MS (ESI+, M+H): 491.3.

### Example 9

### N-(2-{4′-[(1R,3′S)-5,6-Difluoro-3-oxo-3H-spiro[2-benzofuran-1,4′-piperidin]-3′-yl]-3′-methyl-2-biphenylyl}ethyl)propanamide

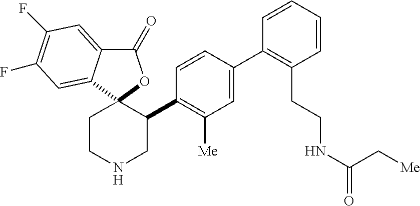

### Step 1: tert-Butyl (1R,3′S)-5,6-difluoro-3′-{3-methyl-2′-[2-(propionylamino)ethyl]-4-biphenylyl}-3-oxo-1′H,3H-spiro[2-benzofuran-1,4′-piperidine]-1′-carboxylate

To a dichloromethane solution (0.05 M) of tert-butyl (1R,3′S)-3′-[2′-(2-aminoethyl)-3-methyl-4-biphenylyl]-5,6-difluoro-3-oxo-1′H,3H-spiro[2-benzofuran-1,4′-piperidine]-1′-carboxylate (1 eq., Example 8, Step 8) was added at 0° C. triethylamine (1.4 eq.) followed by propionyl chloride (1.15 eq.). The resulting reaction mixture was stirred at RT for 1.5 h before it was quenched with the addition of 10% aq. HCl. The aqueous layer was separated and back-extracted with dichloromethane. The combined organic extracts were dried over Na₂SO₄, filtered and the filtrate concentrated in vacuo. Purification by way of column chromatography (SiO₂, Hex→3:2 (v/v) Hex: EtOAc) afforded the title compound.

### Step 2: N-(2-{4′-[(1R,3′S)-5,6-Difluoro-3-oxo-3H-spiro[2-benzofuran-1,4′-piperidin]-3′-yl]-3′-methyl-2-biphenylyl}ethyl)propanamide

To a CH₂Cl₂ solution (0.036 M) of tert-butyl (1R,3′S)-5,6-difluoro-3′-{3-methyl-2′-[2-(propionylamino)ethyl]-4-biphenylyl}-3-oxo-1′H,3H-spiro[2-benzofuran-1,4′-piperidine]-1′-carboxylate (1 eq.) from the previous step was added HCl (4.0 M dioxane solution, 30 eq.). The resulting solution was stirred at RT for 1.5 h. The volatiles were then removed in vacuo to afford the title compound as an HCl salt. MS (ESI+, M+H): 505.3.

### Example 10

### Methyl (2-{4′-[(1R,3′S)-5,6-Difluoro-3-oxo-3H-spiro[2-benzofuran-1,4′-piperidin]-3′-yl]-3′-methyl-2-biphenylyl}ethyl)carbamate

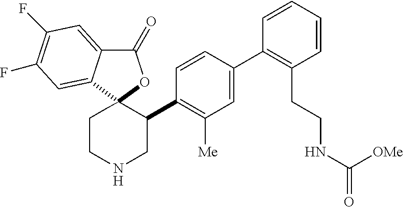

### Step 1: tert-Butyl (1R,3′S)-5,6-difluoro-3′-(2′-{2-[(methoxycarbonyl)amino]ethyl}-3-methyl-4-biphenylyl)-3-oxo-1′H,3H-spiro[2-benzofuran-1,4′-piperidine]-1′-carboxylate

To a dichloromethane solution (0.05 M) of tert-butyl (1R,3′S)-3′-[2′-(2-aminoethyl)-3-methyl-4-biphenylyl]-5,6-difluoro-3-oxo-1′H,3H-spiro[2-benzofuran-1,4′-piperidine]-1′-carboxylate (1 eq., Example 8, Step 8) was added at 0 triethylamine (1.4 eq.) followed by methyl chloroformate (1.15 eq.). The resulting reaction mixture was stirred at 0° C. for 1 h before it was quenched with the addition of 10% aq. HCl. The aqueous layer was separated and back-extracted with dichloromethane. The combined organic extracts were dried over Na₂SO₄, filtered and the filtrate concentrated in vacuo. Purification by way of column chromatography (SiO₂, Hex→3:2 (v/v) Hex: EtOAc) afforded the title compound.

### Step 2: Methyl (2-{4′-[(1R,3′S)-5,6-Difluoro-3-oxo-3H-spiro[2-benzofuran-1,4′-piperidin]-3′-yl]-3′-methyl-2-biphenylyl}ethyl)carbamate

To a CH₂Cl₂ solution (0.036 M) of tert-butyl (1R,3′S)-5,6-difluoro-3′-(2′-{2-[(methoxycarbonyl)amino]ethyl}-3-methyl-4-biphenylyl)-3-oxo-1′H,3H-spiro[2-benzofuran-1,4′-piperidine]-1′-carboxylate (1 eq.) from the previous step was added HCl (4.0 M dioxane solution, 30 eq.). The resulting solution was stirred at RT for 2 h. The volatiles were then removed in vacuo to afford the title compound as an HCl salt. MS (ESI+, M+H): 507.3.

### Example 11

### N-(2-{4′-[(1R,3′S)-5,6-Difluoro-3-oxo-3H-spiro[2-benzofuran-1,4′-piperidin]-3′-yl]-3′-methyl-2-biphenylyl}ethyl)-2-hydroxyacetamide

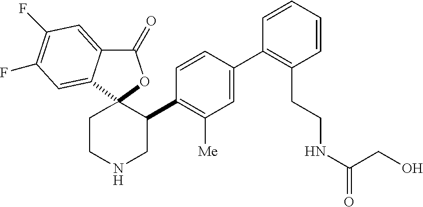

### Step 1: tert-Butyl (1R,3′S)-3′-[2′-(2-[(acetyloxy)acetyl]amino}ethyl)-3-methyl-4-biphenylyl]-5,6-difluoro-3-oxo-1′H,3H-spiro[2-benzofuran-1,4′-piperidine]-1′-carboxylate

To a DMF solution (0.05 M) of tert-butyl (1R,3′S)-3′-[2′-(2-aminoethyl)-3-methyl-4-biphenylyl]-5,6-difluoro-3-oxo-1′H,3H-spiro[2-benzofuran-1,4′-piperidine]-1′-carboxylate (1 eq., Example 8, Step 8) was added acetoxyacetic acid (1.1 eq.), Hunig's base (3 eq.) and HATU (1.2 eq.). The resulting solution was allowed to stir at RT for 20 h before water and EtOAc was added. The aqueous layer was separated and back-extracted with EtOAc. The combined organic extracts were dried over Na₂SO₄, filtered and the filtrate concentrated in vacuo. Purification by way of column chromatography (SiO₂, Hex→3:7 (v/v) Hex: EtOAc) afforded the title compound.

### Step 2: tert-Butyl (1R,3′S)-5,6-difluoro-3′-{2′-[2-(glycoloylamino)ethyl]-3-methyl-4-biphenylyl}-3-oxo-1′H,3H-spiro[2-benzofuran-1,4′-piperidine]-1′-carboxylate

To a methanol solution (0.015 M) of tert-butyl (1R,3′5)-3′-[2′-(2-{[(acetyloxy)acetyl]amino}ethyl)-3-methyl-4-biphenylyl]-5,6-difluoro-3-oxo-1′H,3H-spiro[2-benzofuran-1,4′-piperidine]-1′-carboxylate (1 eq.) from the previous step was added at 0° C. potassium carbonate (3 eq.) in one rapid portion. After 10 min, water was added and the reaction mixture was extracted with EtOAc. The combined organic extracts were then dried over Na₂SO₄, filtered and the filtrate concentrated in vacuo. Purification by way of column chromatography (SiO₂, CH₂Cl₂→90:10 (v/v) CH₂Cl₂: 2.0 M NH₃ in MeOH) afforded the title compound.

### Step 3: N-(2-{4′-[(1R,3′S)-5,6-Difluoro-3-oxo-3H-spiro[2-benzofuran-1,4′-piperidin]-3′-yl]-3′-methyl-2-biphenylyl}ethyl)-2-hydroxyacetamide

To a CH₂Cl₂ solution (0.028 M) of tert-butyl (1R,3′5)-5,6-difluoro-3′-{2′-[2-(glycoloylamino)ethyl]-3-methyl-4-biphenylyl}-3-oxo-1′H,3H-spiro[2-benzofuran-1,4′-piperidine]-1′-carboxylate (1 eq.) from the previous step was added HCl (4.0 M dioxane solution, 40 eq.). The resulting solution was stirred at RT for 3 h. Following the removal of the volatiles in vacuo, the resulting residue was directly subjected to purification by way of column chromatography (SiO₂, 93:7 (v/v) CH₂Cl₂: 2.0 M NH₃ in MeOH 90:10 (v/v) CH₂Cl₂: 2.0 M NH₃ in MeOH) to afford the title compound as a white foam. MS (ESI+, M+H): 507.2. ¹H NMR δ (ppm, CDCl₃): 7.44-7.36 (3H, m), 7.29-7.21 (3H, m), 7.14 (1H, d, J=7.4 Hz), 7.07 (1H, d, J=7.9 Hz), 6.86 (1H, s), 6.41 (1H, s), 4.13-4.01 (2H, m), 3.77 (1H, d, J=11.40 Hz), 3.60-3.48 (2H, m), 3.44-3.23 (5H, m), 2.80-2.72 (1H, m), 2.54-2.37 (4H, m), 2.20 (3H, s), 1.95 (1H, d, J=14.0 Hz). Human Renin IC₅₀ (buffer): 0.66 nM. Human Renin IC₅₀ (plasma): 0.82 nM

### Example 12

### N-(2-{4′-[(1R,3′S)-5-fluoro-6-methoxy-3-oxo-3H-spiro[2-benzofuran-1,4′-piperidin]-3′-yl]-3′-methyl-2-biphenylyl}ethyl)-2-hydroxyacetamide

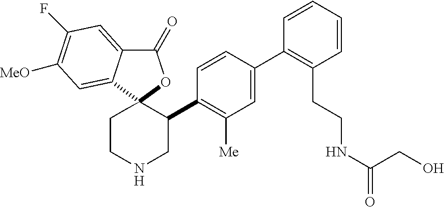

### Step 1: tert-Butyl (1R,3′S)-5-fluoro-3′-{2′-[2-(glycoloylamino)ethyl]-3-methyl-4-biphenylyl}-6-methoxy-3-oxo-1′H,3H-spiro[2-benzofuran-1,4′-piperidine]-1′-carboxylate

To a methanol solution (0.1 M) of tert-butyl (1R,3′S)-3′-[2′-(2-{[(acetyloxy)acetyl]amino}ethyl)-3-methyl-4-biphenylyl]-5,6-difluoro-3-oxo-1′H,3H-spiro[2-benzofuran-1,4′-piperidine]-1′-carboxylate (1 eq., Example 11, Step 1) was added potassium carbonate (3 eq.) in one rapid portion. After heating for 2 h at reflux, water was added and the reaction mixture was extracted with EtOAc. The combined organic extracts were then dried over Na₂SO₄, filtered and the filtrate concentrated in vacuo. Purification by way of column chromatography (SiO₂, CH₂Cl₂→90:10 (v/v) CH₂Cl₂: 2.0 M NH₃ in MeOH) afforded the title compound.

### Step 2: N-(2-{4′-[(1R,3′S)-5-fluoro-6-methoxy-3-oxo-3H-spiro[2-benzofuran-1,4′-piperidin]-3′-yl]-3′-methyl-2-biphenylyl}ethyl)-2-hydroxyacetamide

To a CH₂Cl₂ solution (0.013 M) of tert-butyl (1R,3′S)-5-fluoro-3′-{2′-[2-(glycoloylamino)ethyl]-3-methyl-4-biphenylyl}-6-methoxy-3-oxo-1′H,3H-spiro[2-benzofuran-1,4′-piperidine]-F-carboxylate (1 eq.) from the previous step was added HCl (4.0 M dioxane solution, 40 eq.). The resulting solution was stirred at RT for 3 h. Following the removal of the volatiles in vacuo, the resulting residue was directly subjected to purification by way of column chromatography (SiO₂, 93:7 (v/v) CH₂Cl₂: 2.0 M NH₃ in MeOH→90:10 (v/v) CH₂Cl₂: 2.0 M NH₃ in MeOH) to afford the title compound as a white foam. MS (ESI+, M+H): 519.3.

### Example 13

### N-[2-(2-(4-Bromo-3-[(1R,3′R)-5,6-difluoro-3H-spiro[2-benzofuran-1,4′-piperidin]-3′-yl]-5-isoxazolyl)phenyl)ethyl]acetamide

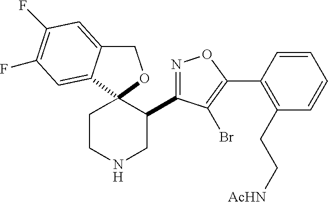

### Step 1: [(2-Bromo-4,5-difluorobenzyl)oxy](tert-butyl)dimethylsilane

To a DMF solution (0.42 M) of (2-bromo-4,5-difluorophenyl)methanol (1 eq., Grignard 1, Step 1) from the previous step and tert-butyl(chloro)dimethylsilane (1.1 eq.) was added imidazole (1.5 eq.) in one rapid portion. The resulting solution was stirred at RT for 14 h before the crude reaction mixture was diluted with hexanes and washed sequentially with water, 10% aq. HCl, 2 N aq. NaOH, water and brine. The organic extract was dried over Na₂SO₄, filtered and the filtrate concentrated in vacuo to furnish the title compound as a colorless oil.

### Step 2: 1-tert-Butyl 3-ethyl 4-[2-({[tert-butyl(dimethyl)silyl]oxy}methyl)-4,5-difluorophenyl]-5,6-dihydro-1,3(2H)-pyridinedicarboxylate

To a 2:1 (v/v) toluene: ethanol solution (0.15 M) of 1-(1,1-dimethylethyl) 3-ethyl 4-(4,4,5,5-tetramethyl-1,3,2-dioxaborolan-2-yl)-5,6-dihydro-1,3(2H)-pyridinedicarboxylate (1 eq.) and [(2-bromo-4,5-difluorobenzyl)oxy](tert-butyl)dimethylsilane (1.2 eq.) from the previous step was added sodium carbonate (2 M aq. solution, 3 eq.). The suspension was evacuated and back-filled with N₂. Finally, [1,1′-bis(diphenylphosphino)ferrocene]dichloropalladium(II) (0.03 eq.) was added in one rapid portion and the reaction suspension was heated at 80° C. for 14 h. The reaction was then quenched with the addition of ether and sat. aq. NH₄Cl. The aqueous layer was separated and back-extracted with ether. The combined organic extracts were washed further with water and brine, dried over Na₂SO₄, filtered and the filtrate concentrated in vacuo. Purification of the crude product thus obtained by way of column chromatography (SiO₂, Hex→1:1 (v/v) Hex: EtOAc) afforded the title compound as a colorless oil that solidified upon standing.

### Step 3: 1′-tert-Butyl 3′-ethyl (1R,3′S)-5,6-difluoro-1′H,3H-spiro[2-benzofuran-1,4′-piperidine]-1′,3′-dicarboxylate

To a THF solution (0.11 M) of 1-tert-butyl 3-ethyl 4-[2-({[tert-butyl(dimethyl)silyl]oxy}methyl)-4,5-difluorophenyl]-5,6-dihydro-1,3(2H)-pyridinedicarboxylate (1 eq.) from the previous step was added TBAF (1 M THF solution, 1.5 eq.). The resulting mixture was then stirred at RT for 2 h. The volatiles were subsequently removed in vacuo and the resulting residue was partitioned between ether and 10% aq. HCl. The organic layer was separated and washed further with water and brine, dried over Na₂SO₄, filtered and the filtrate concentrated in vacuo. Purification of the crude product thus obtained by way of column chromatography (SiO₂, 9:1 (v/v) Hex: EtOAc→3:7 (v/v) Hex: EtOAc) and subsequent chiral separation (Chiralpak AD, 80:10:10:0.25 (v/v/v/v) Hex: MeOH: iPrOH: triethylamine, slower eluting fraction) afforded the title compound as a white solid.

### Step 4: tert-Butyl (1R,3′R)-5,6-difluoro-3′-(hydroxymethyl)-1′H,3H-spiro[2-benzofuran-1,4′-piperidine]-1′-carboxylate

To a toluene solution (0.2 M) of 1′-tert-butyl 3′-ethyl (1R,3′S)-5,6-difluoro-1′H,3H-spiro[2-benzofuran-1,4′-piperidine]-1′,3′-dicarboxylate (1 eq.) from the previous step was added at −78° C. diisobutylaluminum hydride (1.5 M toluene solution, 2.4 eq.). The resulting solution was stirred at −78° C. for 2 h before it was quenched with a sat. aq. solution of Rochelle's salt. The aqueous fraction was separated and back-extracted with EtOAc. The combined organic extracts were washed with water and brine, dried over MgSO₄, filtered and the filtrate concentrated in vacuo. Purification by way of column chromatography (SiO₂, 4:1 (v/v) Hex: EtOAc→3:7 (v/v) Hex: EtOAc) afforded the title compound as a white solid.

### Step 5: tert-Butyl (1R,3′S)-5,6-difluoro-3′-formyl-1′H,3H-spiro[2-benzofuran-1,4′-piperidine]-1′-carboxylate

To a dichloromethane solution (0.05 M) of tert-butyl (1R,3′R)-5,6-difluoro-3′-(hydroxymethyl)-1′H,3H-spiro[2-benzofuran-1,4′-piperidine]-1′-carboxylate (1 eq.) from the previous step was added pyridine (20 eq.) and DMP (1.2 eq.) in one rapid portion. The resulting solution was stirred at RT for 4 h before it was quenched with 10% aq. NaHCO₃. The aqueous layer was separated and back-extracted with dichloromethane. The combined organic extract was then dried over MgSO₄, filtered, and the filtrate concentrated in vacuo. Purification of the crude product thus obtained by way of column chromatography (SiO₂, 4:1 (v/v) Hex: EtOAc→EtOAc) afforded the title compound as a white solid.

### Step 6: tert-Butyl (1R,3′R)-5,6-difluoro-3′-[(E)-(hydroxyimino)methyl]-1′H,3H-spiro[2-benzofuran-1,4′-piperidine]-1′-carboxylate

To an ethanol solution (0.1 M) of tert-butyl (1R,3′S)-5,6-difluoro-3′-formyl-1′H,3H-spiro[2-benzofuran-1,4′-piperidine]-1′-carboxylate (1 eq.) from the previous step and hydroxylamine hydrochloride salt (2 eq.) was added sodium carbonate (2.1 eq.). The resulting mixture was allowed to stir at RT for 3 h before it was diluted with water and ether. The aqueous layer was separated and back-extracted with ether. The combined organic extracts were dried over MgSO₄ and filtered. Concentration of the filtrate in vacuo afforded the title compound as a colorless glass.

### Step 7: N-(2-{2 [(trimethylsilyl)ethynyl]phenyl}ethyl)acetamide

Copper(I) iodide (0.05 eq.), trans-bis(triphenylphosphine) palladium(II) chloride (0.05 eq.), N-[2-(2-bromophenyl)ethyl]acetamide (1 eq.), triethylamine (5 eq.) and trimethylsilylacetylene (2.5 eq.) were taken up in DMF (0.35 M) in a high-pressure tube. The vessel was sealed and the reaction mixture was heated to 100° C. for 72 h. The resulting black suspension was then diluted with ether and washed further with water and brine. The ether extract was dried over Na₂SO₄, filtered and the filtrate concentrated in vacuo. Purification of the crude product thus obtained by way of column chromatography (SiO₂, 9:1 (v/v) Hex: EtOAc→EtOAc) afforded the title compound as a brown oil.

### Step 8: N-{2-[2-(bromoethynyl)phenyl]ethyl}acetamide

To an acetone solution (0.1 M) of silver nitrate (0.3 eq.) and N-(2-{2[(trimethylsilyl)ethynyl]phenyl}ethyl)acetamide (1 eq.) from the previous step was added N-bromosuccinimide (1.2 eq.) portionwise over 10 min. The resulting suspension was stirred at RT for 1 h. The volatiles were then removed in vacuo and the crude product thus obtained was directly subjected to purification by way of column chromatography (SiO₂, 9:1 (v/v) Hex: EtOAc→EtOAc). The title compound was isolated as a tan solid.

### Step 9: tert-Butyl (1R,3′R)-3′-(5-{2-[2-(acetylamino)ethyl]phenyl}-4-bromo-3-isoxazolyl)-5,6-difluoro-1′H,3H-spiro[2-benzofuran-1,4′-piperidine]-1′-carboxylate

To a methanol solution (0.1 M) of tert-butyl (1R,3′R)-5,6-difluoro-3′-[(E)-(hydroxyimino)methyl]-1′H,3H-spiro[2-benzofuran-1,4′-piperidine]-1′-carboxylate (1 eq., Step 6) was added chloramine-T (1 eq.) in one rapid portion. The resulting solution was allowed to stir at RT for 5 min before N-{2-[2-(bromoethynyl)phenyl]ethyl}acetamide (1.2 eq., Step 8) was added. After heating at 80° C. for 16 h, the volatiles were removed in vacuo and the crude product thus obtained was directly subjected to purification by way of column chromatography (SiO₂, 9:1 (v/v) Hex: EtOAc→EtOAc). The title compound was isolated as a white solid.

### Step 10: N-[2-(2-(4-Bromo-3-[(1R,3′R)-5,6-difluoro-3H-spiro[2-benzofuran-1,4′-piperidin]-3′-yl]-5-isoxazolyl)phenyl)ethyl]acetamide

To a CH₂Cl₂ solution (0.05 M) of tert-butyl (1R,3′R)-3′-(5-{2-[2-(acetylamino)ethyl]phenyl}-4-bromo-3-isoxazolyl)-5,6-difluoro-1′H,3H-spiro[2-benzofuran-1,4′-piperidine]-1′-carboxylate (1 eq.) from the previous step was added HCl (4.0 M dioxane solution, 40 eq.). The resulting solution was stirred at RT for 3 h. Following the removal of the volatiles in vacuo, the resulting residue was directly subjected to purification by way of column chromatography (SiO₂, 94:6 (v/v) CH₂Cl₂: 2.0 M NH₃ in MeOH→90:10 (v/v) CH₂Cl₂: 2.0 M NH₃ in MeOH) to afford the title compound as a white foam. MS (ESI+, M+H): 532.1. ¹H NMR δ (ppm, CD₃OD): 7.52-7.47 (1H, m), 7.43-7.31 (3H, m), 7.23 (1H, d, J=7.7 Hz), 7.09-7.05 (1H, m), 5.01 (1H, d, J=12.8 Hz), 4.93 (1H, d, J=12.9 Hz), 3.59-3.47 (2H, m), 3.26-3.09 (5H, m), 2.70-2.62 (2H, m), 2.50-2.45 (1H, m), 2.20-2.14 (1H, m), 1.94-1.82 (4H, m). Human Renin IC₅₀ (buffer): 0.26 nM. Human Renin IC₅₀ (plasma): 1.0 nM

### Example 14

### Assays Demonstrating Biological Activity

### Inhibition of Human Recombinant Renin

Human recombinant renin (Proteos) in 50 mM MOPS pH 7.4, 100 mM NaCl, 0.002% Tween 20 at a final concentration of 100 pM is incubated with inhibitors from a 50 fold concentrated DMSO solution and 6 μM of an internally-quench fluorescent peptide: DNP-Lys-His-Pro-Phe-His-Leu-Val-Ile-His-D,L-Amp (SEQ ID NO: 1); Paschalidou K. et al., Biochem J, 2004, 382, 1031). The reactions take place in a Costar 384 well black plate (#3573) at 37° C. for 3 hours. Fluorescence is measured at times 0 and 3 hours with a SpectraMax Gemini EM reader set at an excitation wavelength of 328 nm and at an emission wavelength of 388 nm. Background fluorescence at t=0 is subtracted from the measurement at t=3 hours. Inhibitory activity of the compounds is expressed as IC₅₀.

### Inhibition of Renin in Human Plasma

Human EDTA-collected plasma is rapidly thawed in warm water and centrifuged at 2900 g for 15 minutes at 4° C. The supernatant is collected and recombinant renin (Proteos) is added at a final concentration of 1 nM. The plasma is transferred to a Costar black 384 well plate (#3573). Renin inhibitors are added from a 17.5 fold concentrated DMSO solution and pre-incubated at 37° C. for 10 minutes. The internally-quench fluorescent peptide QX1520™-Lys-His-Pro-Phe-His-Leu-Val-Ile-His-Lys (5-FAM) (Anaspec) is diluted in 3M Tris pH 7.2, 200 mM EDTA and added to the plasma. The final concentrations are: 6 μM substrate, 342 mM Tris, 23 mM EDTA. The plate is incubated at 37° C. for 1 hour. The plate is read in a SpectraMax Gemini EM reader set at an excitation wavelength of 490 nm and an emission wavelength of 520 nM at times 0 and 1 hour. Background fluorescence at t=0 is subtracted from the measurement at t=1 hour. Inhibitory activity of the compounds is expressed as IC₅₀.

### In Vivo Animal Model

Female double transgenic rats were purchased from RCC Ltd, Füllingsdorf, Switzerland. All animals were maintained under identical conditions and had free access to normal pelleted rat chow and water. Rats were initially treated with enalapril (1 mg/kg/day) during 2 months. After approximately two weeks following cessation of enalapril treatment the double transgenic rats become hypertensive and reach mean arterial blood pressures in the range of 160-170 mmHg.

Transmitter implantation—The rats were anaesthetized with a mixture of 90 mg/kg ketamine-HCl (Ketavet, Parke-Davis, Berlin FRG) and 10 mg/kg xylazine (Rompun, Bayer, Leverkusen, FRG) i.p. The pressure transmitter was implanted under aseptic conditions into the peritoneal cavity with the sensing catheter placed in the descending aorta below the renal arteries pointing upstream. The transmitter was sutured to the abdominal musculature and the skin closed.

Telemetry-System—Telemetry units were obtained from Data Sciences (St. Paul, Minn.). The implanted sensor consisted of a fluid-filled catheter (0.7 mm diameter, 8 cm long; model TA11PA-C40) connected to a highly stable low-conductance strain-gauge pressure transducer, which measured the absolute arterial pressure relative to a vacuum, and a radio-frequency transmitter. The tip of the catheter was filled with a viscous gel that prevents blood reflux and was coated with an antithrombogenic film to inhibit thrombus formation. The implants (length=2.5 cm, diameter=1.2 cm) weighted 9 g and have a typical battery life of 6 months. A receiver platform (RPC-1, Data Sciences) connected the radio signal to digitized input that was sent to a dedicated personal computer (Compaq, deskpro). Arterial pressures were calibrated by using an input from an ambient-pressure reference (APR-1, Data Sciences). Systolic, mean and diastolic blood pressure was expressed in millimeter of mercury (mmHg).

Hemodynamic measurements—Double transgenic rats with implanted pressure transmitters were dosed by oral gavage with vehicle or 10 mg/kg of the test substance (n=6 per group) and the mean arterial blood pressure was continuously monitored. The effect of the test substance is expressed as maximal decrease of mean arterial pressure (MAP) in the treated group versus the control group.

### Results

Compounds in accordance herewith were active, exhibiting an IC50<1 μM in the renin buffer assay. Data for some representative compounds can be found in Examples 4, 5, 11 and 13 above.

What is claimed is:

1. A compound of formula I: 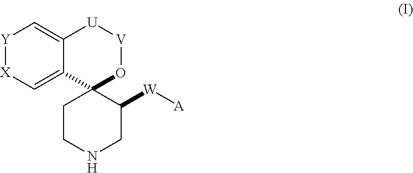
or a stereoisomer thereof, or a pharmaceutically acceptable salt thereof, or a pharmaceutically acceptable salt of the stereoisomer thereof, wherein:
n, for each instance in which it occurs, is independently 0, 1, or 2;
W is a five- or six-membered saturated or unsaturated heterocyclic or carbocyclic monocyclic ring,
wherein the heterocyclic ring contains 1-3 heteroatoms independently selected from N, O and S, wherein each N is optionally in the form of an oxide and each S is optionally in the form of an oxide selected from the group consisting of S(═O) and S(═O)₂,
wherein the heterocyclic or carbocyclic ring is optionally substituted by 1-4 radicals independently selected from the group consisting of:
1) halogen,
2) cyano,
3) R¹,
4) —(CH₂)₀₋₃C(O)R¹,
5) —(CH₂)₀₋₃OR¹,
6) —C(O)OR¹,
7) —(CH₂)₁₋₃CN,
8) —(CH₂)₁₋₃NHC(O)R¹,
9) —(CH₂)₁₋₃NHC(O)OR¹,
10) —(CH₂)₁₋₃NHC(O)(CH₂)₁₋₃OR¹,
wherein substituents (3)-(10) can be further optionally substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or Z, wherein Z is morpholine, oxomorpholine, pyrrolidine, succinimide, acylmorpholine, or thiomorpholine 1,1-dioxide;
A is (i) a five- or six-membered saturated or unsaturated heterocyclic or carbocyclic monocyclic ring or (ii) a first five- or six-membered saturated or unsaturated heterocyclic or carbocyclic ring which is fused to a second five- or six-membered saturated or unsaturated heterocyclic or carbocyclic ring,
wherein the heterocyclic ring in (i), the first heterocyclic ring in (ii) which is fused to the second heterocyclic ring or carbocyclic ring, and the second heterocyclic ring in (ii) to which is fused the first heterocyclic or carbocyclic ring, contain 1-3 heteroatoms independently selected from N, O and S, wherein each N is optionally in the form of an oxide and each S is optionally in the form of an oxide selected from the group consisting of S(═O) and S(═O)₂,
wherein the heterocyclic ring and carbocyclic ring of (i), the first heterocyclic ring and first carbocyclic ring of (ii) and the second heterocyclic ring and second carbocyclic ring of (ii) are independently optionally substituted by 1-4 radicals independently selected from the group consisting of:
1) halogen,
2) cyano,
3) R¹,
4) —(CH₂)₀₋₃C(O)R¹,
5) —(CH₂)₀₋₃OR¹,
6) —C(O)OR¹,
7) —(CH₂)₁₋₃ CN,
8) —(CH₂)₁₋₃NHC(O)R¹,
9) —(CH₂)₁₋₃NHC(O)OR¹,
10) —(CH₂)₁₋₃NHC(O)(CH₂)₁₋₃OR¹,
wherein substituents (3)-(10) can be further optionally substituted with 1-3 halogens, cyano, OR², N(R²)(R³), C(═O)N(R²)(R³), N(R²)C(═O)R³, S(═O)ₙR², S(═O)ₙN(R²)(R³), N(R²)S(═O)ₙR³, aryl, heteroaryl or Z,
wherein Z is morpholine, oxomorpholine, pyrrolidine, succinimide, acylmorpholine, or thiomorpholine 1,1-dioxide;
R² is hydrogen, C₁₋₄ alkyl, C₁₋₄ alkanoyl or C₃₋₆ cycloalkyl, wherein said C₁₋₄ alkyl, C₁₋₄ alkanoyl or C₃₋₆ cycloalkyl group can be independently substituted with 1-3 halogens;
R¹ and R³ are independently selected from the group consisting of R³ hydrogen, C₁₋₄ alkyl, C₂₋₆ alkenyl and C₃₋₆ cycloalkyl, wherein said C₁₋₄ alkyl, C₂₋₆ alkenyl and C₃₋₆ cycloalkyl group can be independently substituted with 1-3 halogens;
V is —(C═O)—, —CH₂— or ═CH—;
U is
a bond or ═CH₂—, or,
when V is ═CH—, U is —CH—;
X is ═CH—, ═CF—, ═C(OR³)—, or —(C═O)—; and
Y is ═CH—, ═CF—, ═N—, or, for the case when X is —(C═O)—, Y is —N(R³)—.

2. The compound of claim 1 having formula Ia: 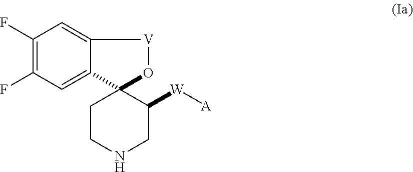
or a pharmaceutically acceptable salt thereof.

3. The compound of claim 1, wherein W is selected from the group consisting of 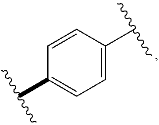
which is unsubstituted or substituted with C₁₋₄ alkyl, and 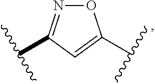
which is unsubstituted or substituted with halogen,
or a pharmaceutically acceptable salt thereof.

4. The compound of claim 1 wherein A is substituted or unsubstituted aryl, or a pharmaceutically acceptable salt thereof.

5. The compound of claim 1 wherein A is aryl, which is unsubstituted or substituted with 1-4 radicals selected from the group consisting of:
1) —CH₂OC(CH₃)₃,
2) —CH₂OH,
3) —CH₂CN,
4) —CH₂CH₂NHC(O)CH₃,
5) —(CH₂)₃OCH₃,
6) —CH₂CH₂C(O)CH₃,
7) —CH₃,
8) —CH₂CH₂NHC(O)CH₂CH₃,
9) —CH₂CH₂NHC(O)OCH₃, and
10) —CH₂CH₂NHC(O)CH₂OH
or a pharmaceutically acceptable salt thereof.

6. A compound of claim 1, selected from the group consisting of
rac-(1R,3′S)3′-[2′-(tert-Butoxymethyl)-3-methyl-4-biphenylyl]-5,6-Difluoro-3H-spiro[2-benzofuran-1,4′-piperidine],
rac-{4′-[(1R,3′S)-5,6-Difluoro-3H-spiro[2-benzofuran-1,4′-piperidin]-3′-yl]-3′-methyl-2-biphenylyl}methanol,
rac-{4′-[(1R,3′S)-5,6-Difluoro-3H-spiro[2-benzofuran-1,4′-piperidin]-3′-yl]-3′-methyl-2-biphenylyl}acetonitrile,
rac-N-(2-{4′-[(1R,3′S)-5,6-Difluoro-3H-spiro[2-benzofuran-1,4′-piperidin]-3′-yl]-3′-methyl-2-biphenylyl}ethyl)acetamide,
rac-(1R,3′S)-3′-[2′-(3-Methoxypropyl)-3-methyl-4-biphenylyl]-5-methyl-3,5-dihydro-6H-spiro[furo[3,4-c]pyridine-1,4′-piperidin]-6-one,
rac-(1R,3′S)-5-methyl-3′-[3-methyl-2′-(3-oxobutyl)-4-biphenylyl]-3,5-dihydro-6H-spiro[furo[3,4-c]pyridine-1,4′-piperidin]-6-one,
rac-N-(2-{4′-[(1R,3′S)-5,6-Difluoro-3H-spiro[2-benzofuran-1,4′-piperidin]-3′-yl]-3′,6-dimethyl-2-biphenylyl}ethyl)acetamide,
N-(2-{4′-[(1R,3′S)-5,6-Difluoro-3-oxo-3H-spiro[2-benzofuran-1,4′-piperidin]-3′-yl]-3′-methyl-2-biphenylyl}ethyl)acetamide,
N-(2-{4′-[(1R,3′S)-5,6-Difluoro-3-oxo-3H-spiro[2-benzofuran-1,4′-piperidin]-3′-yl]-3′-methyl-2-biphenylyl}ethyl)propanamide,
Methyl (2-{4′-[(1R,3′S)-5,6-Difluoro-3-oxo-3H-spiro[2-benzofuran-1,4′-piperidin]-3′-yl]-3′-methyl-2-biphenylyl}ethyl)carbamate,
N-(2-{4′-[(1R,3′S)-5,6-Difluoro-3-oxo-3H-spiro[2-benzofuran-1,4′-piperidin]-3′-yl]-3′-methyl-2-biphenylyl}ethyl)-2-hydroxyacetamide,
N-(2-{4′-[(1R,3′S)-5-fluoro-6-methoxy-3-oxo-3H-spiro[2-benzofuran-1,4′-piperidin]-3′-yl]-3′-methyl-2-biphenylyl}ethyl)-2-hydroxyacetamide, and
N-[2-(2-(4-Bromo-3-[(1R,3′R)-5,6-difluoro-3H-spiro[2-benzofuran-1,4′-piperidin]-3′-yl]-5-isoxazolyl)phenyl)ethyl]acetamide
or a pharmaceutically acceptable salt thereof.

7. A method of inhibiting renin activity, said method comprising the step of administering a compound according to claim 1 in an amount sufficient to provide an effective amount for renin inhibition in an organism.

8. A pharmaceutical composition comprising as an active ingredient, a compound according to claim 1 in free form or in pharmaceutically acceptable salt form, together with one or more customary pharmaceutical excipients.

9. A method of treating hypertension, congestive heart failure, cardiac hypertrophy, cardiac fibrosis, postinfarction cardiomyopathy, nephropathy, vasculopathy, neuropathy, restenosis following angioplasty, raised intra-ocular pressure, glaucoma, abnormal vascular growth, hyperaldosteronism or anxiety states, comprising the step of administering a therapeutically effective amount of a compound according to claim 1 in the free form or in the form of a pharmaceutically acceptable salt is administered to an organism in need of such treatment.
